SEQUENCE LISTING (7 sequences)

SEQ ID NO: 1 - protein, 13 aa, Artificial Sequence
  peptide
ESAVAESREERMG

SEQ ID NO: 2 - protein, 18 aa, Artificial Sequence
  peptide
QHADSLENIDESAVAESR

SEQ ID NO: 3 - protein, 18 aa, Artificial Sequence
  peptide
RRAASMDNNSKFAKSRSR

SEQ ID NO: 4 - DNA, 876 bp, Homo sapiens
atggagaagctggcggcctctacagagccccaagggcctcggccggtcctgggccgtgagagtgtccaggtgcccgatgaccaagactttcgcagcttccggtcagagtgtgaggctgaggtgggctggaacctgacctatagcagggctggggtgtctgtctgggtgcaggctgtggagatggatcggacgctgcacaagatcaagtgccggatggagtgctgtgatgtgccagccgagacactctacgacgtcctacacgacattgagtaccgcaagaaatgggacagcaacgtcattgagacttttgacatcgcccgcttgacagtcaacgctgacgtgggctattactcctggaggtgtcccaagcccctgaagaaccgtgatgtcatcaccctccgctcctggctccccatgggcgctgattacatcattatgaactactcagtcaaacatcccaaatacccacctcggaaagacttggtccgagctgtgtccatccagacgggctacctcatccagagcacagggcccaagagctgcgtcatcacctacctggcccaggtggaccccaaaggctccttacccaagtgggtggtgaataaatcttctcagttcctggctcccaaggccatgaagaagatgtacaaggcgtgcctcaagtaccccgagtggaaacagaagcacctgcctcacttcaagccgtggctgcacccggagcagagcccgttgccgagcctggcgctgtcggagctgtcggtgcagcatgcggactcactggagaacatcgacgagagcgcggtggccgagagcagagaggagcggatgggcggcgcgggcggcgagggcagcgacgacgacacctcgctcacctga

SEQ ID NO: 5 - protein, 291 aa, Homo sapiens
MEKLAASTEPQGPRPVLGRESVQVPDDQDFRSFRSECEAEVGWNLTYSRAGVSVWVQAVEMDRTLHKIKCRMECCDVPAETLYDVLHDIEYRKKWDSNVIETFDIARLTVNADVGYYSWRCPKPLKNRDVITLRSWLPMGADYIIMNYSVKHPKYPPRKDLVRAVSIQTGYLIQSTGPKSCVITYLAQVDPKGSLPKWVVNKSSQFLAPKAMKKMYKACLKYPEWKQKHLPHFKPWLHPEQSPLPSLALSELSVQHADSLENIDESAVAESREERMGGAGGEGSDDDTSLT

SEQ ID NO: 6 - protein, 359 aa, Homo sapiens
MSTRAKKLRRIWRILEEEESVAGAVQTLLLRSQEGGVTSAAASTLSEPPRRTQESRTRTRALGLPTLPMEKLAASTEPQGPRPVLGRESVQVPDDQDFRSFRSECEAEVGWNLTYSRAGVSVWVQAVEMDRTLHKIKCRMECCDVPAETLYDVLHDIEYRKKWDSNVIETFDIARLTVNADVGYYSWRCPKPLKNRDVITLRSWLPMGADYIIMNYSVKHPKYPPRKDLVRAVSIQTGYLIQSTGPKSCVITYLAQVDPKGSLPKWVVNKSSQFLAPKAMKKMYKACLKYPEWKQKHLPHFKPWLHPEQSPLPSLALSELSVQHADSLENIDESAVAESREERMGGAGGEGSDDDTSLT

SEQ ID NO: 7 - protein, 214 aa, Homo sapiens
MELAAGSFSEEQFWEACAELQQPALAGADWQLLVETSGISIYRLLDKKTGLHEYKVFGVLEDCSPTLLADIYMDSDYRKQWDQYVKELYEQECNGETVVYWEVKYPFPMSNRDYVYLRQRRDLDMEGRKIHVILARSTSMPQLGERSGVIRVKQYKQSLAIESDGKKGSKVFMYYFDNPGGQIPSWLINWAAKNGVPNFLKDMARACQNYLKKT

(19) United States
(12) Patent Application Publication
(10) Pub. No.: US 2006/0148032 A1
(43) Pub. Date: Jul. 6, 2006

(54) Novel phosphoprotein

(75) Inventors: Monilola Olayioye, Ulm (DE); Jane Visvader, Kew (AU); Geoffrey Lindeman, Kew (AU); Peter Hoffmann, Adelaide (AU); Thomas Pomorski, Berlin (DE)

Correspondence Address: KNOBBE MARTENS OLSON & BEAR LLP, 2040 MAIN STREET, FOURTEENTH FLOOR, IRVINE, CA, 92614, US

(73) Assignee: The Walter and Eliza Hall Institute of Medical Res, Parkville (AU)

(21) Appl. No.: 10/538,704

(22) PCT Filed: Dec. 12, 2003
(86) PCT No.: PCT/AU03/01664

§ 371 Date: Feb. 21, 2006

(30) Foreign Application Priority Data
Dec. 13, 2002 (AU) 2002953341

(51) Int. Cl. C07K 14/82; C07H 21/04; C12P 21/06

(52) U.S. Cl. 435/69.1; 435/320.1; 435/325; 530/352; 536/23.5

(57) ABSTRACT

The present invention relates generally to cancer therapy and cancer diagnostics and to agents useful therefor. More particularly, the present invention provides a novel tumorigenesis-related phosphoprotein and a nucleic acid molecule encoding same. The present invention also provides diagnostic agents to detect the presence or absence of the tumorigenesis-related phosphoprotein or the presence or absence of an expressible tumorigenesis-related gene encoding the phosphoprotein. Such diagnostic agents are useful in determining the likelihood of development of a tumor in a vertebrate animal such as mammal and, in particular, a human. The diagnostic agents provided by the present invention may be used inter alia in screening and/or predicting the likelihood of development of neoplastic diseases such as but not limited to mammary cancer.

### BACKGROUND OF THE INVENTION

1. Field of the Invention

The present invention relates generally to cancer therapy and cancer diagnostics and to agents useful therefor. More particularly, the present invention provides a novel tumorigenesis-related phosphoprotein and a nucleic acid molecule encoding same. The present invention also provides diagnostic agents to detect the presence or absence of the tumorigenesis-related phosphoprotein or the presence or absence of an expressible tumorigenesis-related gene encoding the phosphoprotein. Such diagnostic agents are useful in determining the likelihood of development of a tumor in a vertebrate animal such as mammal and, in particular, a human. The diagnostic agents provided by the present invention may be used inter alia in screening and/or predicting the likelihood of development of neoplastic diseases such as but not limited to mammary cancer.

2. Description of the Prior Art

Bibliographic details of the publications referred to by author in this specification are collected at the end of the description.

Reference to any prior art in this specification is not, and should not be taken as, an acknowledgment or any form of suggestion that this prior art forms part of the common general knowledge in any country.

The increasing sophistication of recombinant DNA technology is greatly facilitating research and development in the medical and allied health industries. This is particularly the case in relation to genetic and immunologically based diagnostic assays for disease conditions or for determining the propensity for disease conditions to develop.

The genetic bases behind cancer initiation and development are complex and generally involve a network of multiple dominant, positive regulators of oncogenesis (i.e. oncogenes) as well as multiple negative regulators, sometimes referred to as tumor suppressor genes, including, for example, transcription factors and cell-cycle regulators. The genetic complexity of tumor initiation and development requires that further research be conducted to enable the generation of a comprehensive library of oncogenes and tumor suppressor genes to enable a full diagnosis or therapeutic regimen to be implemented. This is particularly the case since it is considered unlikely that a mutation in a single gene will prove to be responsible for the development of all tumors.

The receptor tyrosine kinase ErbB2 was first identified as the mammalian counterpart of the v-erbB oncogene and belongs to the family of ErbB receptors, comprising four members (HER1/EGFR (epidermal growth factor receptor), HER2/ErbB2/Neu, HER3/ErbB3 and HER4/ErbB4). These receptors signal cooperatively by forming ligand-induced combinations of homo- and heterodimers. Over-expression of ErbB1 and ErbB2 is associated with a variety of human cancers. Of particular significance are the amplification and/or over-expression of ErbB2 in up to 30% of human breast cancer patients, in which it correlates with poor prognosis.

The importance of the ErbB2 signaling in mammary tumorigenesis has been established using mouse models. Over-expression of Neu, driven off the mouse mammary tumor virus promoter, induced focal mammary tumors which frequently metastasized to the lung (Guy et al., Proc. Natl. Acad. Sci. USA 89: 10578-10582, 1992). The long latency period, however, suggested that additional molecular events were required for tumors to develop. Further analysis of these tumors identified somatic mutations in neu, resulting in constitutive activation of the receptor. The up-regulation of the ErbB3 protein concomitant with an increase in ErbB3 tyrosine phosphorylation in these tumors suggested that ErbB2 synergizes with ErbB3 in oncogenesis. The ErbB2/ErbB3 heterodimer has been shown to promote efficient activation of the PI3K pathway, a pathway essential for cell proliferation and survival. However, other kinases implicated in tumorigenesis such as Src and Erk are also activated downstream of this ErbB heterodimer. Although downstream targets of ErbB signaling have been characterized, the crucial molecular players with regard to Neu-induced transformation, specifically transcription factors that execute the signals received, remain elusive.

The ability to detect tumors or a likelihood or predisposition of development of a tumor would be of great benefit in terms of patient care and initiating anti-tumor therapy at an early stage.

The regulation of the FoxO subgroup of forkhead transcription factors, consisting of forkhead (FKH) transcription factors R (FKHR), RL1 (FkHRL1) and AFX has been recently identified as being via the PI3K pathway. These proteins drive the expression of cell cycle regulators, such as p27 and apoptotic proteins including Bim and Fas ligand. Phosphorylation on specific serine and threonine residues by phosphokinase B (PKB) leads to their inactivation and sequestration in the cytoplasm by 14-3-3 scaffolding proteins, thereby preventing their negative control on cell proliferation.

In accordance with the present invention, a novel 35 kD phosphoprotein has been identified which is specifically recognized by a phospho-FKHR antibody. It is proposed that the novel protein is a useful diagnostic and prognostic target for cancer as well as being a validated drug target.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A summary of the sequence identifiers is provided in Table 1. A sequence listing is provided at the end of the specification.

The aberrant expression of the ErbB2 (also known as HER2 and Neu) receptor tyrosine kinase, implicated in the pathogenesis of a high proportion of sporadic breast cancers, was investigated. Targeted expression of ErbB2 to the mammary gland in mice leads to tumor development. A long latency period, however, suggested that additional molecular alterations were acquired during the transformation process. A novel phosphoprotein, referred to herein as “FORS” (Forkhead Related Serine) was identified as being up-regulated at the mRNA level in Neu tumors and in human breast carcinoma cell lines. The FORS protein is now referred to as StarD10. The gene (including cDNA) encoding StarD10 is referred to herein as “StarD10”. The terms “StarD10” and “FORS” and “StarD10” and “Fors” may be used interchangeably.

It is proposed, in accordance with the present invention, that alterations in the StarD10 protein, the StarD10 gene or the expression of the StarD10 gene facilitates development of, or otherwise predisposes a subject to, the development of a tumor and in particular a mammary tumor. The term “tumor” includes all forms of cancer including sarcomas and carcinomas. The present invention further relates to the treatment or prophylaxis of a tumor and in particular breast cancer by the application of genetic therapy or the administration of proteinaceous or non-proteinaceous molecules which mimic or otherwise obviate the need for or otherwise act as agonists of StarD10 or StarD10. Furthermore, StarD10 or StarD10 or derivatives or homologs thereof, may be employed to screen for potential therapeutic agents such as from natural sources or chemical or peptide libraries.

It is further proposed, therefore, that StarD10 or StarD10 represents a validated drug target for modulators of StarD10 expression or StarD10 activity. In addition, StarD10 binds to lipids and has a role in cellular phospholipid signalling.

Another aspect of the present invention provides an isolated nucleic acid molecule comprising a nucleotide sequence of StarD10. Reference to “StarD10” include genomic and cDNA molecules. Such nucleic acid molecules may be useful in genetic therapy to modulate expression of genomic StarD10 and/or recombinant StarD10 and, in particular, to reduce or otherwise ameliorate production of StarD10.

The present invention further provides methods of diagnosis of a tumor, and in particular a breast cancer, by detecting presence or levels of StarD10 and/or mutations therein, or aberrations in one or both alleles of the StarD10 gene, or in the expression of one or both gene alleles. The present invention provides, therefore, diagnostic agents such as primers or probes of the StarD10 gene or mutated portions thereof. The diagnostic agents of the present invention extend to immunointeractive molecules and in particular antibodies to all or antigenic parts of StarD10. Such diagnostic agents are useful in the detection of a tumor or a predisposition for development of a tumor and in particular breast cancer.

### BRIEF DESCRIPTION OF THE FIGURES

FIG. 1 is a representation of polyacrylamide gel electrophoresis (PAGE) showing that a tumor-specific 35 kD protein cross-reacts with a phospho-FKHR antibody. (A) Mammary tumors (T) and corresponding adjacent tissue (AT) or normal mammary gland tissue (NT) from B17, C13 and B14 MMTV-Neu transgenic female were dissected, protein lysates were prepared and separated by SDS PAGE. The gel was blotted and probed with phospho-FKHR specific antibody. After stripping, the gel was re-probed with (B) ErbB2- and (C) tubulin-specific antibodies. Lysate from insulin-stimulated 293T cells, transiently transfected with a FKHR expression construct, was loaded as a positive control.

FIG. 2 presents diagrammatic representations showing the purification of the 35 kD protein from SKBR3 cytosol. SKBR3 cytosolic protein lysate was subjected to (A) free flow electrophoresis and (B) reverse phase HPLC as described in the Examples. The fractions obtained were separated by SDS PAGE and analyzed by immunoblotting using phospho-FKHR specific antibody. In the case of RP-HPLC samples, two fractions were pooled. (C) Coomassie stain of fractions containing the 35 kD protein from SKBR3 cytosolic protein lysate separated by (i) FFE, (ii) RP HPLC and (iii) 10% w/v SDS PAGE. (D) Alignment of part of the sequence of Q9Y365 (SEQ ID NO:2; band #1 in (C)) with part of the sequence of FKHR1 (SEQ ID NO:3) around serine 256 (underlined), using the Dialign software.

FIG. 3 provides representations indicating that StarD10 is a phosphoprotein and that it is highly expressed in liver. (A) Amino acid sequence of the full-length Q9Y365 (SEQ ID NO:6). Peptides identified by mass spectrometry are highlighted. Methionine 68, from which translation is initiated, is indicated. In (B) and (C), 293T cells were transiently transfected with HA- or Flag-tagged StarD10. (B) Lysates were prepared and separated by SDS PAGE and immunoblotted using phospho-FKHR specific antibody. SKBR3 protein lysate was loaded as a control. (C) StarD10 was immunoprecipitated with Flag antibody and incubated without or with alkaline phosphatase (CIP) for 45 min at 37° C. prior to separation by SDS PAGE. The gel was blotted and probed with phospho-FKHR-specific antibody and re-probed with Flag specific antibody. 293T whole cell extract (WCE) containing transfected Flag StarD10 was loaded as a control. (D) A commercially available membrane (Clontech) containing poly(A)⁺ RNA from different human tissues was hybridized with a StarD10 cDNA probe.

FIG. 4 provides representations of Northern analysis showing over-expression of StarD10 in breast and colon carcinoma cell lines. (A) Poly(A)⁺ RNA from breast epithelial and (B) 20 μg total RNA from colon carcinoma cell lines were hybridized with StarD10 and ErbB2 or EGFR cDNA probes, respectively. Equal loading was confirmed by hybridizing with a GAPDH cDNA probe. Note than only 6 μg RNA was loaded in the case of SKBR3 cells.

FIG. 5 presents diagrammatic representations showing StarD10 expression and localization using a highly specific purified peptide antibody. (A) Alignment of human Pctp (SEQ ID NO:7) and StarD10 (SEQ ID NO:5). The start domain is shown in red; the peptide to which a StarD10-specific antibody was raised to is highlighted in blue. In (B) and (C), protein lysates of cell lines or mouse liver, as indicated, were separated by SDS PAGE. Gels were immunoblotted with purified StarD10-specific antibodies #419 and/or #420. In (C), membranes were re-probed with ErbB2, phospho-FKHR and tubulin specific antibodies. (D) Merged confocal microscopy images of MCF7 cells immunostained with #420 StarD10-specific antibody (green) and counterstained with DAPI (blue). The control panel shows staining with secondary FITC-conjugated antibody and DAPI alone.

FIG. 6 provides representations indicating the growth properties of murine fibroblasts stably expressing StarD10. (A) Protein lysates of NIH3T3 and HER14 cells infected with pBabePuro and pBabeStarD10 retroviral expression vectors, respectively, were separated by SDS PAGE and immunoblotted with Pctp1 antibody. (B) 2000 cells per well (96 well plate) were plated on day 0 in duplicates in 10% v/v and 0.5% v/v serum. Cell growth was assayed by addition of MTS reagent and measurement of absorbance after 2 hours at 490 nm, as described in the Examples.

FIG. 7 shows photographic representation indicating anchorage independent growth of fibroblasts expressing StarD10. (A) 50,000 NIH3T3 Puro and StarD10 cells, and (B) and (C) 2,000 HER14 Puro and StarD10 cells were plated with or without 10 ng/ml EGF in a 0.35% w/v top agar. Photographs were taken 4 weeks later after staining with NBT overnight at a 2-fold magnification in (A) and a 1.5 fold magnification in (B) and (C).

FIG. 8A-H is a photographic representation showing staining of breast carcinomas for StarD10.

FIG. 9 is a photographic and graphic representation showing expression of StarD10 in HER14 cells.

FIG. 10 is a graphical representation showing the effects of donor liposomes containing TNP-PE and pyrene-C₁₂-PC or pyrene-C₁₂-PE mixed with a 20-fold excess of unlabeled acceptor liposomes. After 60 sec, purified StarD10 protein was added and pyrene fluorescence at 395 nm was recorded (excitation: 340 nm). Spectra were normalized for maximum fluorescence in Tirton. The emission spectrum of pyrene overlaps with the excitation of TNP, resulting in FRET when lipid analogs are in close proximity. The increase in fluorescence is evidence for transfer of pyrene-labeled lipid analogs to acceptor liposomes.

A summary of sequence identifiers used throughout the subject specification is provided in Table 1.

TABLE 1
Summary of sequence identifiers

| SEQUENCE | | |
|---|---|---|
| ID NO. | NAME | DESCRIPTION |
| 1 | StarD10 | partial amino acid sequence of the |
| | 265-277 | StarD10 phosphoprotein |
| | | (ESAVAESREERMG)¹ |
| 2 | Q9Y365 | partial amino acid sequence of a |
| | | predicted human protein based on a |
| | | comparative screen of the |
| | | C. elegans proteome |
| | | (QHADSLENIDESAVAESR)¹ |
| 3 | FKHR1 | partial amino acid sequence of the |
| | | transcription factor, FKHR |
| | | (RRAASMDNNSKFAKSRSR)¹ |
| 4 | StarD10 | full-length cDNA encoding the |
| | | StarD10 phosphoprotein |
| 5 | StarD10 | full-length amino acid sequence of |
| | | StarD10 phosphoprotein |
| 6 | full | full-length amino acid sequence of |
| | Q9Y365 | predicted human protein Q9Y365 |
| 7 | PCTP | full-length amino acid sequence of |
| | | human phosphatidylcholine transfer |
| | | protein PCTP |

¹single amino acid code (see Table 2 for definitions)

Single and three letter abbreviations used throughout the specification are defined below.

TABLE 2
Single and three letter amino acid abbreviations

| | THREE-LETTER | ONE-LETTER |
|---|---|---|
| AMINO ACID | ABBREVIATION | SYMBOL |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any residue | Xaa | X |

### DETAILED DESCRIPTION OF THE INVENTION

The present invention provides an isolated nucleic acid molecule comprising a sequence of nucleotides encoding a tumorigenesis-related phosphoprotein involved in cellular phospholipid signalling. The gene of the present invention is referred to herein as “StarD10”. The tumorigenesis-related proteinaceous expression product of StarD10 is referred to herein as StarD10. The expression product may, however, also include RNA (including spliced introns and exons) and an mRNA transcript and these are encompassed by the term “StarD10”. This term includes a gene or a cDNA molecule. A proteinaceous product includes a polypeptide comprising two or more amino acid residues. A “polypeptide” in this context includes a protein or peptide. The present invention provides isolated nucleic acid molecules corresponding to StarD10 as well as nucleic acid primers and probes useful for detecting StarD10, aberrations in the StarD10 gene including deletions, insertions, additions and point mutations as well as gene silencing events. The present invention further provides a means for detecting StarD10 expression products including polypeptides and fragments thereof. The present invention further provides genetically modified animals having one or both alleles of StarD10 or its homolog deleted, mutated or its/their expression silenced. The present invention further provides genetically modified animals comprising StarD10 genes introduced or deleted. Such animals are useful animal models for testing the pathogenesis of cancer and/or for testing potential anti-cancer agents.

Accordingly, one aspect of the present invention provides an isolated nucleic acid molecule comprising a sequence of nucleotides encoding or complementary to a sequence encoding the amino acid sequence set forth in SEQ ID NO:5 or SEQ ID NO:6 or an amino acid sequence having at least about 40% similarity to SEQ ID NO:5 or SEQ ID NO:6 after optimal alignment.

As stated above, SEQ ID NO:5 represents a polypeptide expression product of StarD10 and is included in the term “StarD10”. SEQ ID NO:6 represents the amino acid sequence of a predicted human protein, based on comparative screen of the C. elegans proteome with human EST nucleotide databases, designated herein “full Q9Y365”.

StarD10 is proposed, in accordance with the present invention, to be a tumorigenesis-related phosphoprotein that is over-expressed in mouse mammary tumor cells and in human breast carcinoma cell lines, and is furthermore shown herein to cooperate with the ErbB/HER2 receptor in facilitating cellular transformation. The isolated nucleic acid molecule encoding the tumorigenesis-related phosphoprotein StarD10 is referred to herein as StarD10. As stated above, the term “StarD10” includes a genomic gene, a cDNA molecule and an mRNA transcript.

In a more particular aspect of the present invention, there is provided an isolated nucleic acid molecule comprising a sequence of nucleotides encoding or complementary to a sequence encoding a polypeptide capable of facilitating, increasing or otherwise promoting the development of a tumor in a vertebrate animal, said polypeptide comprising an amino acid sequence as set forth in SEQ ID NO:5 or SEQ ID NO:6 or an amino acid sequence having at least about 40% similarity to SEQ ID NO:5 or SEQ ID NO:6 after optimal alignment.

As stated above, the isolated nucleic acid molecule is referred to herein as StarD10.

Consequently, an even more particular aspect of the present invention contemplates an isolated nucleic acid molecule comprising a nucleotide sequence or a complementary nucleotide sequence having the identifying characteristics of StarD10 including a nucleotide sequence substantially as set forth in SEQ ID NO:4 or a part or fragment thereof or a nucleotide sequence having at least about 40% similarity to SEQ ID NO:4 after optimal alignment or a nucleotide sequence capable of hybridizing to SEQ ID NO:4 or its complementary form under low stringency conditions.

Reference to “StarD10” includes a genomic DNA, cDNA or mRNA sequence comprising an open reading frame or a genomic nucleotide sequence comprising exons and optionally introns.

Reference herein to “StarD10” includes derivatives or homologs thereof. A derivative includes a mutant, part, fragment or portion of StarD10 as well as any fusion derivatives such as formed by the fusion of one or more nucleotide sequences to the 5′ or 3′ terminal portion of StarD10 or to the 5′ or 3′ terminal portion of a fragment of StarD10 such as an intron or exon fragment. A derivative of StarD10 includes single or multiple nucleotide substitutions, additions and/or deletions or inversions to all or part of StarD10 and also includes point mutations and cross-over events. The term “StarD10” also includes its 5′ and 3′ untranslated regions including a promoter operably linked to StarD10 or a heterologous gene.

The present invention provides, therefore, an isolated nucleic acid molecule capable of directing transcription of a second nucleic acid molecule operably linked to the first mentioned nucleic acid molecule wherein said first nucleic acid molecule comprises a nucleotide sequence which is 5′ of a nucleotide sequence set forth in SEQ ID NO:4 or a nucleotide sequence having at least about 40% similarity to SEQ ID NO:4 after optimal alignment or a nucleotide sequence capable of hybridizing to SEQ ID NO:4 or its complement under low stringency conditions.

In this embodiment, the first mentioned nucleic acid molecule is referred to herein as the StarD10 promoter. The StarD10 promoter may be used to direct expression of StarD10 or of a heterologous gene or cDNA molecule.

The StarD10 gene may be from an animal cell including from a mammal, insect, reptile, fish, avian species, arachnid or lower order organism such as a yeast, fungus or C. elegans. Most preferably, the StarD10 gene is from a mammal such as a human or other primate, livestock animal (eg. sheep, pig, cow, donkey, horse, goat), laboratory test animal (e.g. mouse, rat, rabbit, hamster, guinea pig), a companion animal (e.g. dog, cat) or a captive wild animal. Most preferably, the StarD10 gene is from a human or mouse.

The exemplified StarD10 nucleotide sequence is of human origin and more particularly for human breast carcinoma cell line SKBR3 available from ATCC under Catalog No. HTB30. Reference herein to “StarD10” includes reference to the human forms of the gene as well as homologs from other species such as C. elegans as well as a range of polymorphic variants and/or splice variants.

The term “gene” is used in its broadest sense and includes cDNA corresponding to the exons of a gene. Accordingly, reference herein to a “gene” is to be taken to include:
(i) a classical genomic gene consisting of transcriptional and/or translational regulatory sequences and/or a coding region and/or non-translated sequences (i.e. introns, 5′- and 3′-untranslated sequences); or
(ii) mRNA or cDNA corresponding to the coding regions (i.e. exons) and 5′- and 3′-untranslated sequences of the gene.

The term “gene” is also used to describe synthetic or fusion molecules encoding all or part of an expression product. In particular embodiments, the term “nucleic acid molecule” and “gene” may be used interchangeably. The term “polynucleotide” may also be used to describe a nucleic acid molecule, nucleotide sequence and a gene.

The term “similarity” as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In a particularly preferred embodiment, nucleotide and sequence comparisons are made at the level of identity rather than similarity.

Terms used to describe sequence relationships between two or more polynucleotides or polypeptides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units, inclusive of nucleotides and amino acid residues, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as, for example, disclosed by Altschul et al. (Nucl. Acids Res. 25: 3389, 1997). A detailed discussion of sequence analysis can be found in Unit 19.3 of Ausubel et al. (“Current Protocols in Molecular Biology” John Wiley & Sons Inc, 1994-1998, Chapter 15).

The terms “sequence similarity” and “sequence identity” as used herein refer to the extent that sequences are identical or functionally or structurally similar on a nucleotide-by-nucleotide basis or an amino acid-by-amino acid basis over a window of comparison. Thus, a “percentage of sequence identity”, for example, is calculated by comparing two optimally aligned sequences over the window of comparison, determining the number of positions at which the identical nucleic acid base (eg. A, T, C, G, I) or the identical amino acid residue (eg. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

Preferably, the percentage similarity between a particular sequence and a reference sequence (nucleotide or amino acid) is at least about 50% or at least about 60% or at least about 70% or at least about 80% or at least about 90% or at least about 95% or above such as at least about 96%, 97%, 98%, 99% or greater.

Reference herein to a low stringency includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C) % (Marmur and Doty, J. Mol. Biol. 5: 109, 1962). However, the Tₘ of a duplex DNA decreases by 1° C. with every increase of 1% in the number of mismatch base pairs (Bonner and Laskey, Eur. J. Biochem. 46: 83, 1974). Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

A “StarD10 allele” refers to normal alleles of the StarD10 locus as well as alleles carrying variations that may modulate an individual's predisposition to the development of a tumor and, in particular, a mammary tumour.

The terms “nucleic acids”, “nucleotide” and “polynucleotide” include RNA, cDNA, genomic DNA, synthetic forms and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (eg. methyl phosphonates, phosphotriesters, phosphoamidates, carbamates, etc.), charged linkages (eg. phosphorothioates, phosphorodithioates, etc.), pendent moieties (eg. polypeptides), intercalators (eg. acridine, psoralen, etc.), chelators, alkylators and modified linkages (eg. α-anomeric nucleic acids, etc.). Also included are synthetic molecules that mimic polynucleotides in their ability to bind to a designated sequence via hydrogen binding and other chemical interactions. Such molecules are known in the art and include, for example, those in which peptide linkages substitute for phosphate linkages in the backbone of the molecule.

The present invention further provides recombinant nucleic acids including a recombinant construct comprising all or part of the StarD10 coding and/or regulatory region. The recombinant construct may be capable of replicating autonomously in a host cell. Alternatively, the recombinant construct may become integrated into the chromosonal DNA of the host cell. Such a recombinant polynucleotide comprises a polynucleotide of genomic, cDNA, semi-synthetic or synthetic origin which, by virtue of its origin or manipulation: (i) is not associated with all or a portion of a polynucleotide with which it is associated in nature; (ii) is linked to a polynucleotide other than that to which it is linked in nature; or (iii) does not occur in nature. Where nucleic acids according to the invention include RNA, reference to the sequence shown should be construed as reference to the RNA equivalent with U substituted for T.

Therefore, recombinant nucleic acids comprising sequences otherwise not naturally occurring are provided by the present invention. Although the wild-type sequence may be employed, it will often be altered, eg. by deletion, substitution or insertion.

cDNA or genomic libraries of various types may be screened as natural sources of the nucleic acids of the present invention or such nucleic acids may be provided by amplification of sequences resident in genomic DNA or other natural sources, eg. by PCR. The choice of cDNA libraries normally corresponds to a tissue source, which is abundant in mRNA for the desired protein. Phage libraries are normally preferred but other types of libraries may be used. Clones of a library are spread onto plates, transferred to a substrate for screening, denatured and probed for the presence of desired sequences.

As used herein, the terms “StarD10 locus” and “StarD10 allele” refer to the double-stranded DNA comprising the locus, allele or region, as well as either of the single-stranded DNAs comprising the locus, allele or region.

As used herein, a “portion or part or fragment” of the StarD10 locus or allele is defined as having a minimal size of at least about 10 nucleotides or preferably about 13 nucleotides or more preferably at least about 20 nucleotides and may have a minimal size of at least about 40 nucleotides. This definition includes all sizes in the range of 10-40 nucleotides as well as greater than 40 nucleotides. Thus, this definition includes nucleic acids of 12, 15, 20, 25, 40, 60, 80, 100, 200, 300, 400, 500 or 700 nucleotides or nucleic acids having any number of nucleotides within these values (eg. 13, 16, 23, 30, 38, 50, 72, 121, 673 etc. nucleotides) or nucleic acids having more than 700 nucleotides or any number of nucleotides between 700 and the number shown in SEQ ID NO:4. The present invention includes all novel nucleic acids having at least 10 nucleotides derived from SEQ ID NO:4, or a complement or functional equivalent thereof.

“Operably linked” refers to a juxtaposition wherein the components so described are in a relationship permitting them to function in their intended manner. For instance, a promoter is operably linked to a coding sequence if the promoter effects its transcription or expression.

Large amounts of the polynucleotides of the present invention may be produced by replication in a suitable host cell. Natural or synthetic polynucleotide fragments coding for a desired fragment will be incorporated into recombinant polynucleotide constructs, usually DNA constructs, capable of introduction into and replication in a prokaryotic or eukaryotic cell. Usually, the polynucleotide constructs are suitable for replication in a unicellular host, such as yeast or bacteria, but may also be intended for introduction to (with or without integration within the genome) cultured mammalian or plant or other eukaryotic cell lines. The purification of nucleic acids produced by the methods of the present invention are described, eg. in Sambrook et al. (Molecular Cloning: A Laboratory Manual, 2^{nd} Ed., Cold Spring Harbor Laboratory, Cold Spring Harbor, N.Y., 1989).

The polynucleotides of the present invention may also be produced by chemical synthesis, eg. by the phosphoramidite method described by Beaucage and Carruthers (Tetra. Letts. 22: 1859-1862, 1981) or the triester method according to Matteucci et al. (J. Am. Chem. Soc. 103: 3185, 1981) and may be performed on commercial, automated oligonucleotide synthesizers. A double-stranded fragment may be obtained from the single-stranded product of chemical synthesis either by synthesizing the complementary strand and annealing the strands together under appropriate conditions or by adding the complementary strand using DNA polymerase with an appropriate primer sequence.

Polynucleotide constructs prepared for introduction into a prokaryotic or eukaryotic host may comprise a replication system recognized by the host, including the intended polynucleotide fragment encoding the desired polypeptide and will preferably also include transcription and translational initiation regulatory sequences operably linked to the polypeptide encoding segment. Expression vectors may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences and mRNA stabilizing sequences. Secretion signals may also be included, where appropriate, which allow the protein to cross and/or lodge in cell membranes or be secreted from the cell. Such vectors may be prepared by means of standard recombinant techniques well known in the art and discussed, for example, in Sambrook et al. (1989) supra.

An appropriate promoter and other necessary vector sequences is selected so as to be functional in the host and may include, when appropriate, those naturally associated with StarD10 genes. Examples of workable combinations of cell lines and expression vectors are described in Sambrook et al. (1989) supra). Many useful vectors are known in the art and may be obtained from suppliers such as Stratagene, New England Biolabs, Promega Biotech and others. Promoters such as the trp, lac and phage promoters, tRNA promoters and glycolytic enzyme promoters may be used in prokaryotic hosts. Useful yeast promoters include promoter regions for metallothionein, 3-phosphoglycerate kinase or other glycolytic enzymes such as enolase or glyceraldehyde-3-phosphate dehydrogenase, enzymes responsible for maltose and galactose utilization and others. Vectors and promoters suitable for use in yeast expression are further described in European Patent Publication No. 0 073 675. Appropriate non-native mammalian promoters might include the early and late promoters from SV40 (Fiers et al., Nature 273: 113-120, 1978) or promoters derived from murine mammary tumor virus (MMTV), mouse tumor virus, avian sarcoma viruses, adenovirus II, bovine papilloma virus or polyoma. Insect promoters may be derived from baculovirus. In addition, the construct may be joined to an amplifiable gene (eg. DHFR) so that multiple copies of the gene may be made. For appropriate enhancer and other expression control sequences, see also Enhancers and Eukaryotic Gene Expression, Cold Spring Harbor Press, Cold Spring Harbour, N.Y. (1983). See also, eg. U.S. Pat. No. 5,691,198.

While such expression vectors may replicate autonomously, they may also replicate by being inserted into the genome of the host cell, by methods well known in the art.

Expression and cloning vectors will likely contain a selectable marker and a gene encoding a protein necessary for survival or growth of a host cell transformed with the vector. The presence of this gene ensures growth of those host cells that express the inserts. Typical selection genes encode proteins that (a) confer resistance to antibiotics or other toxic substances, eg. ampicillin, neomycin, methotrexate, etc., (b) complement auxotrophic deficiencies, or (c) supply critical nutrients not available from complex media, eg. the gene encoding D-alanine racemase for Bacillus. The choice of the proper selectable marker will depend on the host cell and appropriate markers for different hosts are well known in the art.

The vectors containing the nucleic acids of interest can be transcribed in vitro and the resulting RNA introduced into the host cell by well-known methods, eg. by injection (see Kubo et al., FEBS Lett. 241: 119, 1988), or the vectors can be introduced directly into host cells by methods well known in the art, which vary depending on the type of cellular host, including electroporation; transfection employing calcium chloride, rubidium chloride, calcium phosphate, DEAE-dextran, or other substances; microprojectile bombardment; lipofection; infection (where the vector is an infectious agent, such as a retroviral genome); and other methods. See generally, Sambrook et al. (1989) supra. The introduction of the polynucleotides into the host cell by any method known in the art, including, inter alia, those described above, will be referred to herein as “transfection”. The cells into which have been introduced nucleic acids described above are meant to also include the progeny of such cells.

Large quantities of the nucleic acids and polypeptides (see below) of the present invention may be prepared by expressing the StarD10 nucleic acids or portions thereof in vectors or other expression vehicles in compatible prokaryotic or eukaryotic host cells. The most commonly used prokaryotic hosts are strains of E. coli, although other prokaryotes, such as Bacillus subtilis or Pseudomonas may also be used.

Mammalian or other eukaryotic host cells, such as those of yeast, filamentous fungi, plant, insect or amphibian or avian species, may also be useful for production of the proteins of the present invention. Propagation of mammalian cells in culture is per se well known. See Jakoby and Pastan (eds.), Cell Culture. Methods in Enzymology, Vol. 58, 1979 (Academic Press, Inc., Harcour Brace Jovanovich (N.Y.). Examples of commonly used mammalian host cell lines are VERO and HeLa cells, Chinese hamster ovary (CHO) cells, 293T and NIH3T3 cell lines, and WI38, BHK, and COS cell lines. An example of a commonly used insect cell line is SF9. However, it will be appreciated by the skilled practitioner that other cell lines may be appropriate, eg. to provide higher expression, desirable glycosylation patterns or other features.

Clones are selected by using markers depending on the mode of the vector construction. The marker may be on the same or a different DNA molecule, preferably the same DNA molecule. In prokaryotic hosts, the transformant may be selected, eg. by resistance to ampicillin, tetracycline or other antibiotics. Production of a particular product based on temperature sensitivity may also serve as an appropriate marker.

Prokaryotic or eukaryotic cells transformed with the polynucleotides of the present invention are useful not only for the production of the nucleic acids and polypeptides of the present invention but also, for example, in studying the characteristics of a StarD10 polypeptide.

Antisense polynucleotide sequences are particularly useful in the context of the present invention as they may prevent or diminish the expression of the StarD10 locus and, as will be appreciated by those skilled in the art, this may effect reduction or elimination of expression products from StarD10, including StarD10 mRNA and StarD10. Since StarD10 is over-expressed in breast cancer cell lines and in mammary tumors, and is associated with the undesirable changes concomitant with cellular transformation and the development of uncontrolled proliferation, the means to ameliorate or diminish StarD10 may have beneficial therapeutic potential. Polynucleotide vectors, for example, containing all or a portion of the StarD10 locus or other sequences from the StarD10 region (particularly those flanking the StarD10 locus) may be placed under the control of a promoter in an antisense orientation and introduced into a cell. Expression of such an antisense construct within a cell interferes with StarD10 transcription and/or translation. Furthermore, alternative approaches to down-regulation using sense constructs, such as co-suppression and mechanisms to induce RNAi, may also be employed.

The present invention is particularly useful for screening compounds by using the StarD10 polypeptide or binding fragment thereof in any of a variety of drug screening techniques, such as those described herein and in International Publication No. WO 97/02048.

Yet another aspect of the present invention contemplates an expression product of StarD10. The expression product may be a polypeptide encoded by the open reading frame of StarD10 (i.e. see SEQ ID NO:5 or SEQ ID NO:6) or it may be an mRNA or RNA molecule spliced from an mRNA or precursor form thereof. Such RNA molecules include exons and introns. Such molecules or their corresponding DNA sequences may be useful as regulatory molecules in their own right or as diagnostic probes or primers for detecting wild-type or a mutated or deleted StarD10 gene.

Accordingly, another aspect of the present invention provides an expression product from StarD10, said expression product selected from the list comprising:
(i) an amino acid sequence set forth in SEQ ID NO:5 or an amino acid sequence having at least 40% similarity to SEQ ID NO:5 after optimal alignment;
(ii) an amino acid sequence set forth in SEQ ID NO:5;
(iii) an amino acid sequence set forth in SEQ ID NO:6 or an amino acid sequence having at least 40% similarity to SEQ ID NO:6 after optimal alignment;
(iv) an amino acid sequence set forth in SEQ ID NO:6;
(v) a ribonucleotide sequence corresponding to SEQ ID NO:4 or a nucleotide sequence having at least about 40% similarity to SEQ ID NO:4 after optimal alignment or a nucleotide sequence capable of hybridizing to SEQ ID NO:4 or its complementary form under low stringency conditions; and
(vi) a ribonucleotide sequence corresponding to SEQ ID NO:4.

“StarD10 protein” or “StarD10 polypeptide” or specific StarD10 molecules from human or murine or other sources refers to a protein including a polypeptide encoded by the StarD10 locus, variants or fragments thereof. The term “polypeptide” refers to a polymer of amino acids and its equivalent and does not refer to a specific length of the product; thus, peptides, oligopeptides and proteins are included within the definition of a polypeptide. This term also does not refer to or exclude modifications of the polypeptide, for example, glycosylations, aceylations, phosphorylations and the like. Included within the definition are, for example, polypeptides containing one or more analogs of an amino acid (including, for example, unnatural amino acids, etc.), polypeptides with substituted linkages as well as other modifications known in the art, both naturally and non-naturally occurring. Ordinarily, such polypeptides will be at least about 40% similar to the natural StarD10 sequence, preferably in excess of 50% such as greater than 60%, 70%, 80% or 90% and more preferably at least about 95% similar. Also included are proteins encoded by DNAs which hybridize under high or low stringency conditions to StarD10-encoding nucleic acids and closely related polypeptides or proteins retrieved by antisera to the StarD10 protein.

The StarD10 polypeptide is shown in SEQ ID NO:5 and SEQ ID NO:6 and may be in isolated and/or purified form, free or substantially free of material with which it is naturally associated. The polypeptide may, if produced by expression in a prokaryotic cell or produced synthetically, lack native post-translational processing, such as glycosylation. Alternatively, the present invention is also directed to polypeptides which are sequence variants, alleles or derivatives of the StarD10 polypeptide. Such polypeptides may have an amino acid sequence which differs from that set forth in SEQ ID NO:5 or SEQ ID NO:6 by one or more of addition, substitution, deletion or insertion of one or more amino acids. Conveniently, such polypeptides have StarD10 function but if not, they may nevertheless be useful in diagnostic or therapeutic assays.

Substitutional variants typically contain the exchange of one amino acid for another at one or more sites within the protein and may be designed to modulate one or more properties of the polypeptide such as stability against proteolytic cleavage without the loss of other functions or properties. Amino acid substitutions may be made on the basis of similarity in polarity, charge, solubility, hydrophobicity, hydrophilicity and/or the amphipathic nature of the residues involved. Preferred substitutions are ones that are conservative, that is, one amino acid is replaced with one of similar shape and charge. Conservative substitutions are well known in the art and typically include substitutions within the following groups: glycine, alanine; valine, isoleucine, leucine; aspartic acid, glutamic acid; asparagine, glutamine; serine, threonine; lysine, arginine; and tyrosine, phenylalanine.

Certain amino acids may be substituted for other amino acids in a protein structure without appreciable loss of interactive binding capacity with structures such as, for example, antigen-binding regions of antibodies or binding sites on substrate molecules or binding sites on proteins interacting with the StarD10 polypeptide. Since it is the interactive capacity and nature of a protein which defines that protein's biological functional activity, certain amino acid substitutions can be made in a protein sequence and its underlying DNA coding sequence and nevertheless obtain a protein with like properties. In making such changes, the hydropathic index of amino acids may be considered. The importance of the hydrophobic amino acid index in conferring interactive biological function on a protein is generally understood in the art (Kyte and Doolittle, J. Mol. Biol. 157: 105-132, 1982).

Alternatively, the substitution of like amino acids can be made effectively on the basis of hydrophilicity. The importance of hydrophilicity in conferring interactive biological function of a protein is generally understood in the art (U.S. Pat. No. 4,554,101). The use of the hydrophobic index or hydrophilicity in designing polypeptides is further discussed in U.S. Pat. No. 5,691,198.

The length of the polypeptide sequences compared for homology will generally be at least about 16 amino acids, usually at least about 20 residues, more usually at least about 24 residues, typically at least about 28 residues and preferably more than about 35 residues.

The present invention further contemplates chemical analogs of the StarD10 polypeptide.

Analogs contemplated herein include but are not limited to modification to side chains, incorporating of unnatural amino acids and/or their derivatives during peptide, polypeptide or protein synthesis and the use of crosslinkers and other methods which impose conformational constraints on the proteinaceous molecule or their analogues.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid, contemplated herein is shown in Table 3.

TABLE 3
Codes for non-conventional amino acids

| Non-conventional | |
|---|---|
| amino acid | Code |
| α-aminobutyric acid | Abu |
| α-amino-α-methylbutyrate | Mgabu |
| aminocyclopropane- | Cpro |
| carboxylate | |
| aminoisobutyric acid | Aib |
| aminonorbornyl- | Norb |
| carboxylate | |
| cyclohexylalanine | Chexa |
| cyclopentylalanine | Cpen |
| D-alanine | Dal |
| D-arginine | Darg |
| D-aspartic acid | Dasp |
| D-cysteine | Dcys |
| D-glutamine | Dgln |
| D-glutamic acid | Dglu |
| D-histidine | Dhis |
| D-isoleucine | Dile |
| D-leucine | Dleu |
| D-lysine | Dlys |
| D-methionine | Dmet |
| D-ornithine | Dorn |
| D-phenylalanine | Dphe |
| D-proline | Dpro |
| D-serine | Dser |
| D-threonine | Dthr |
| D-tryptophan | Dtrp |
| D-tyrosine | Dtyr |
| D-valine | Dval |
| D-α-methylalanine | Dmala |
| D-α-methylarginine | Dmarg |
| D-α-methylasparagine | Dmasn |
| D-α-methylaspartate | Dmasp |
| D-α-methylcysteine | Dmcys |
| D-α-methylglutamine | Dmgln |
| D-α-methylhistidine | Dmhis |
| D-α-methylisoleucine | Dmile |
| D-α-methylleucine | Dmleu |
| D-α-methyllysine | Dmlys |
| D-α-methylmethionine | Dmmet |
| D-α-methylornithine | Dmorn |
| D-α-methylphenylalanine | Dmphe |
| D-α-methylproline | Dmpro |
| D-α-methylserine | Dmser |
| D-α-methylthreonine | Dmthr |
| D-α-methyltryptophan | Dmtrp |
| D-α-methyltyrosine | Dmty |
| D-α-methylvaline | Dmval |
| D-N-methylalanine | Dnmala |
| D-N-methylarginine | Dnmarg |
| D-N-methylasparagine | Dnmasn |
| D-N-methylaspartate | Dnmasp |
| D-N-methylcysteine | Dnmcys |
| D-N-methylglutamine | Dnmgln |
| D-N-methylglutamate | Dnmglu |
| D-N-methylhistidine | Dnmhis |
| D-N-methylisoleucine | Dnmile |
| D-N-methylleucine | Dnmleu |
| D-N-methyllysine | Dnmlys |
| N-methylcyclohexylalanine | Nmchexa |
| D-N-methylornithine | Dnmorn |
| N-methylglycine | Nala |
| N-methylaminoisobutyrate | Nmaib |
| N-(1-methylpropyl)glycine | Nile |
| N-(2-methylpropyl)glycine | Nleu |
| D-N-methyltryptophan | Dnmtrp |
| D-N-methyltyrosine | Dnmtyr |
| D-N-methylvaline | Dnmval |
| γ-aminobutyric acid | Gabu |
| L-t-butylglycine | Tbug |
| L-ethylglycine | Etg |
| L-homophenylalanine | Hphe |
| L-α-methylarginine | Marg |
| L-α-methylaspartate | Masp |
| L-α-methylcysteine | Mcys |
| L-α-methylglutamine | Mgln |
| L-αmethylhistidine | Mhis |
| L-α-methylisoleucine | Mile |
| L-α-methylleucine | Mleu |
| L-α-methylmethionine | Mmet |
| L-α-methylnorvaline | Mnva |
| L-α-methylphenylalanine | Mphe |
| L-α-methylserine | Mser |
| L-α-methyltryptophan | Mtrp |
| L-α-methylvaline | Mval |
| N-(N-(2,2-diphenylethyl) | Nnbhm |
| carbamylmethyl)glycine | |
| 1-carboxy-1-(2,2-diphenyl- | Nmbc |
| ethylamino)cyclopropane | |
| L-N-methylalanine | Nmala |
| L-N-methylarginine | Nmarg |
| L-N-methylasparagine | Nmasn |
| L-N-methylaspartic acid | Nmasp |
| L-N-methylcysteine | Nmcys |
| L-N-methylglutamine | Nmgln |
| L-N-methylglutamic acid | Nmglu |
| L-Nmethylhistidine | Nmhis |
| L-N-methylisolleucine | Nmile |
| L-N-methylleucine | Nmleu |
| L-N-methyllysine | Nmlys |
| L-N-methylmethionine | Nmmet |
| L-N-methylnorleucine | Nmnle |
| L-N-methylnorvaline | Nmnva |
| L-N-methylornithine | Nmorn |
| L-N-methylphenylalanine | Nmphe |
| L-N-methylproline | Nmpro |
| L-N-methylserine | Nmser |
| L-N-methylthreonine | Nmthr |
| L-N-methyltryptophan | Nmtrp |
| L-N-methyltyrosine | Nmtyr |
| L-N-methylvaline | Nmval |
| L-N-methylethylglycine | Nmetg |
| L-N-methyl-t-butylglycine | Nmtbug |
| L-norleucine | Nle |
| L-norvaline | Nva |
| α-methyl-aminoisobutyrate | Maib |
| α-methyl-γ-aminobutyrate | Mgabu |
| α-methylcyclohexylalanine | Mchexa |
| α-methylcylcopentylalanine | Mcpen |
| α-methyl-α-napthylalanine | Manap |
| α-methylpenicillamine | Mpen |
| N-(4-aminobutyl)glycine | Nglu |
| N-(2-aminoethyl)glycine | Naeg |
| N-(3-aminopropyl)glycine | Norn |
| N-amino-α-methylbutyrate | Nmaabu |
| α-napthylalanine | Anap |
| N-benzylglycine | Nphe |
| N-(2-carbamylethyl)glycine | Ngln |
| N-(carbamylmethyl)glycine | Nasn |
| N-(2-carboxyethyl)glycine | Nglu |
| N-(carboxymethyl)glycine | Nasp |
| N-cyclobutylglycine | Ncbut |
| N-cycloheptylglycine | Nchep |
| N-cyclohexylglycine | Nchex |
| N-cyclodecylglycine | Ncdec |
| N-cylcododecylglycine | Ncdod |
| N-cyclooctylglycine | Ncoct |
| N-cyclopropylglycine | Ncpro |
| N-cycloundecylglycine | Ncund |
| N-(2,2-diphenylethyl)glycine | Nbhm |
| N-(3,3-diphenylpropyl)glycine | Nbhe |
| N-(3-guanidinopropyl)glycine | Narg |
| N-(1-hydroxyethyl)glycine | Nthr |
| N-(hydroxyethyl))glycine | Nser |
| N-(imidazolylethyl))glycine | Nhis |
| N-(3-indolylyethyl)glycine | Nhtrp |
| N-methyl-γ-aminobutyrate | Nmgabu |
| D-N-methylmethionine | Dnmmet |
| N-methylcyclopentylalanine | Nmcpen |
| D-N-methylphenylalanine | Dnmphe |
| D-N-methylproline | Dnmpro |
| D-N-methylserine | Dnmser |
| D-N-methylthreonine | Dnmthr |
| N-(1-methylethyl)glycine | Nval |
| N-methyla-napthylalanine | Nmanap |
| N-methylpenicillamine | Nmpen |
| N-(p-hydroxyphenyl)glycine | Nhtyr |
| N-(thiomethyl)glycine | Ncys |
| penicillamine | Pen |
| L-α-methylalanine | Mala |
| L-α-methylasparagine | Masn |
| L-α-methyl-t-butylglycine | Mtbug |
| L-methylethylglycine | Metg |
| L-α-methylglutamate | Mglu |
| L-α-methylhomophenylalanine | Mhphe |
| N-(2-methylthioethyl)glycine | Nmet |
| L-α-methyllysine | Mlys |
| L-α-methylnorleucine | Mnle |
| L-α-methylornithine | Morn |
| L-α-methylproline | Mpro |
| L-α-methylthreonine | Mthr |
| L-α-methyltyrosine | Mtyr |
| L-N-methylhomophenylalanine | Nmhphe |
| N-(N-(3,3-diphenylpropyl) | Nnbhe |
| carbamylmethyl)glycine | |

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogues by introducing covalent bonds such as forming an amide bond between the N and C termini, between two side chains or between a side chain and the N or C terminus.

The term “peptide mimetic” or “mimetic” is intended to refer to a substance, which has the essential biological activity of the StarD10 polypeptide. A peptide mimetic may be a peptide-containing molecule that mimics elements of protein secondary structure (Johnson et al., “Peptide Turn Mimetics” in Biotechnology and Pharmacy, Pezzuto et al., Eds., Chapman and Hall, New York, 1993). The underlying rationale behind the use of peptide mimetics is that the peptide backbone of proteins exists chiefly to orient amino acid side chains in such a way as to facilitate molecular interactions such as those of antibody and antigen, enzyme and substrate or scaffolding proteins. A peptide mimetic is designed to permit molecular interactions similar to the natural molecule. A mimetic may not be a peptide at all, but it will retain the essential biological activity of natural StarD10 polypeptide.

The StarD10 polypeptide or fragment employed in a test such as a diagnostic or therapeutic assay may either be free in solution, affixed to a solid support, or borne on a cell surface. One method of drug screening utilizes eukaryotic or prokaryotic host cells that are stably transformed with recombinant polynucleotides expressing the polypeptide or fragment, preferably in competitive binding assays. Such cells, either in viable or fixed form, can be used for standard binding assays. One may measure, for example, the formation of complexes between a StarD10 polypeptide or fragment and the agent being tested, or examine the degree to which the formation of a complex between a StarD10 polypeptide or fragment and a known ligand is aided or interfered with by the agent being tested.

It is proposed in accordance with the present invention that tumors have a propensity to develop in subjects who have cells in which the StarD10 gene is expressed. Consequently, the diagnostic and prognostic methods of the present invention detect or assess the presence of the StarD10 gene or locus and confirm the presence of a tumor or a predisposition to the development of a tumor. Useful diagnostic techniques to detect the presence of StarD10 include but are not limited to fluorescent in situ hybridization (FISH), direct DNA sequencing, PFGE analysis, Southern blot analysis, single-stranded conformational analysis (SSCA), RNase protection assay, allele-specific oligonucleotide (ASO hybridization), dot blot analysis and single-stranded conformation polymorphism assay (SSCP) [Orita et al., Proc. Nat. Acad. Sci. USA 86: 2776-2770, 1989]. Also useful is DNA microchip technology as well as electrotide. Electrotides may also be used as a detection system. Such a system relies on complementary binding of RNA or DNA to assemble an electronic circuit which thereby creates a detectable electronic signal. One particularly useful system is eSensor (trade mark; Motorola) which is well described at http://www.motorola.com/lifesciences/esensor/tech_overview.htm1.

Predisposition to cancers, such as mammary tumors, can be ascertained by testing any tissue of a human for the presence of an expressible StarD10 gene. For example, an individual who has inherited a germ-line StarD10 gene would be prone to develop cancers. This can be determined by testing DNA from any tissue of an individual's body. In addition, pre-natal diagnosis can be accomplished by testing foetal cells, placental cells or amniotic fluid for the presence of the StarD10 gene.

It should be pointed out that reference to a “subject”, “individual”, “patient” or “vertebrate animal” is not to be taken as limiting the target to a particular animal or human. The present invention is applicable to detecting tumors in a range of vertebrate animals, although humans are preferred.

Alteration of StarD10 mRNA expression can be detected by any of a number of techniques known in the art. These include Northern blot analysis, PCR amplification, RNase protection and microchip technology. Diminished mRNA expression correlates with reduced levels of expressed StarD10 phosphoprotein and, consequently, increased likelihood of enhanced therapeutic outcomes. Analogously, enhanced mRNA expression correlates with enhanced levels of expressed StarD10 phosphoprotein and, consequently, increased likelihood of the existence of or the propensity for the development of cancer and, in particular, mammary cancer. The need for intervention to deliver improved therapeutic outcomes is thereby indicated.

StarD10 genes can also be detected by screening for a StarD10 protein. For example, monoclonal and polyclonal antibodies immunoreactive with StarD10 can be used to screen a tissue. Presence of cognate antigen indicates existence of StarD10 phosphoprotein and, hence, increased chance of the existence of or the propensity for the development of cancer and, especially, breast cancer. Such immunological assays can be done in any convenient format known in the art. These include Western blots, immunohistochemical assays and ELISA assays. Any means for detecting a StarD10 protein can be used to detect a wild-type StarD10 gene.

The use of monoclonal antibodies in an immunoassay is particularly preferred because of the ability to produce them in large quantities and the homogeneity of the product. The preparation of hybridoma cell lines for monoclonal antibody production is derived by fusing an immortal cell line and lymphocytes sensitized against the immunogenic preparation (i.e. comprising StarD10) or can be done by techniques which are well known to those who are skilled in the art. (See, for example, Douillard and Hoffman, Basic Facts about Hybridomas, in Compendium of Immunology Vol. II, ed. by Schwartz, 1981; Kohler and Milstein, Nature 256: 495-499, 1975; Kohler and Milstein, European Journal of Immunology 6: 511-519, 1976).

Another aspect of the present invention contemplates a method for detecting StarD10 in a biological sample from a subject, said method comprising contacting said biological sample with an antibody specific for StarD10 or its derivatives or homologs for a time and under conditions sufficient for an antibody-StarD10 complex to form, and then detecting said complex.

The presence of StarD10 may be accomplished in a number of ways such as by Western blotting and ELISA procedures. A wide range of immunoassay techniques are available as can be seen by reference to U.S. Pat. Nos. 4,016,043, 4,424,279 and 4,018,653. These include both single-site and two-site or “sandwich” assays of the non-competitive types, as well as in the traditional competitive binding assays. These assays also include direct binding of a labeled antibody to a target.

Sandwich assays are among the most useful and commonly used assays and are favoured for use in the present invention. A number of variations of the sandwich assay technique exist, and all are intended to be encompassed by the present invention. Briefly, in a typical forward assay, an unlabeled antibody is immobilized on a solid substrate and the sample to be tested brought into contact with the bound molecule. After a suitable period of incubation, for a period of time sufficient to allow formation of an antibody-antigen complex, a second antibody specific to the antigen, labeled with a reporter molecule capable of producing a detectable signal is then added and incubated, allowing time sufficient for the formation of another complex of antibody-antigen-labeled antibody. As stated above, the antigen is StarD10 or a fragment thereof. Any unreacted material is washed away, and the presence of the antigen is determined by observation of a signal produced by the reporter molecule. The results may either be qualitative, by simple observation of the visible signal, or may be quantified by comparing with a control ample containing known amounts of hapten. Variations on the forward assay include a simultaneous assay, in which both sample and labeled antibody are added simultaneously to the bound antibody. These techniques are well known to those skilled in the art, including any minor variations as will be readily apparent. In accordance with the present invention, the sample is one which might contain StarD10 including cell extract or tissue biopsy. As StarD10 is an intracellular molecule, cell extracts and in particular cytoplasmic extracts are preferred. The sample is, therefore, generally a biological sample comprising biological fluid.

In a typical forward sandwich assay, a first antibody having specificity for the StarD10 or antigenic parts thereof, is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs or microplates, or any other surface suitable for conducting an immunoassay. The binding processes are well known in the art and generally consist of cross-linking covalently binding or physically adsorbing, the polymer-antibody complex to the solid surface, which is then washed in preparation for the test sample. An aliquot of the sample to be tested is then added to the solid phase complex and incubated for a period of time sufficient (eg. 2-40 minutes or overnight if more convenient) and under suitable conditions (eg. from room temperature to about 37° C. including 25° C.) to allow binding of any sub-unit present in the antibody. Following the incubation period, the antibody sub-unit solid phase is washed and dried and incubated with a second antibody specific for a portion of the antigen. The second antibody is linked to a reporter molecule, which is used to indicate the binding of the second antibody to the antigen.

An alternative method involves immobilizing the target molecules in the biological sample and then exposing the immobilized target to specific antibody, which may or may not be labeled with a reporter molecule. Depending on the amount of target and the strength of the reporter molecule signal, a bound target may be detectable by direct labeling with the antibody.

Alternatively, a second labeled antibody, specific to the first antibody is exposed to the target-first antibody complex to form a target-first antibody-second antibody tertiary complex. The complex is detected by the signal emitted by the reporter molecule.

By “reporter molecule”, as used in the present specification, is meant a molecule which, by its chemical nature, provides an analytically identifiable signal which allows the detection of antigen-bound antibody. Detection may be either qualitative or quantitative. The most commonly used reporter molecules in this type of assay are enzymes, fluorophores or radionuclide-containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, β-galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labeled antibody is added to the first antibody hapten complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantified, usually spectrophotometrically, to give an indication of the amount of hapten which was present in the sample. “Reporter molecule” also extends to use of cell agglutination or inhibition of agglutination such as red blood cells on latex beads, and the like.

Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state to excitability in the molecule, followed by emission of the light at a characteristic color visually detectable with a light microscope. As in the EIA, the fluorescent-labeled antibody is allowed to bind to the first antibody-hapten complex. After washing off the unbound reagent, the remaining tertiary complex is then exposed to the light of the appropriate wavelength, the fluorescence observed indicates the presence of the hapten of interest. Immunofluorescence and EIA techniques are both very well established in the art and are particularly preferred for the present method. However, other reporter molecules, such as radioisotope, chemiluminescent or bioluminescent molecules, may also be employed.

The present invention is predicated in part on the determination that aberrant cells and in particular cancer cells and even more particularly mammary cancer cells or cells with a propensity for developing into mammary cells express elevated levels of StarD10 relative to normal cells. StarD10 is a phosphoprotein, which is demonstrated herein to be associated with inter alia cellular transformation. Moreover, its over-expression correlated with the up-regulation of the ErbB/HER2 receptor, over-expression of which in 30% of human breast cancer cases is known to be associated with poor prognosis. Hence, a cell that produces high levels of the StarD10 protein is deemed, in accordance with the present) invention, to be an aberrant cell and a cell associated with or likely to be associated with a disease such as cancer and, in particular, mammary cancer. The ability to detect StarD10, or expression products of genetic material StarD10 which encodes StarD10, provides a means for detecting or diagnosing cancer or a propensity for the development of cancer.

Accordingly, in another aspect, the present invention contemplates a method for detecting an aberrant cell in a subject or in a biological sample from said subject, said method comprising contacting cells or cell extracts from said subject or said biological sample with an immunointeractive molecule specific for StarD10 or an antigenic portion thereof and screening for the level of immunointeractive molecule-StarD10 complex formations wherein an elevated presence of said complex relative to a normal cell is indicative of an aberrant cell.

In a related embodiment, the present invention provides a method for detecting an aberrant cell in a subject or in a biological sample from said subject, said method comprising screening the level of an expression product of a gene encoding a StarD10, wherein an elevated level of said expression product compared to a normal cell is indicative of an aberrant cell.

A “biological sample” from a subject includes a biopsy and may be any sample of cells, cell extract, tissue, tissue fluid, excretia, circulatory fluid or respiratory fluid or other material. The biological sample may be extracted, treated, untreated, diluted or concentrated from the subject.

Reference to a “normal” cell includes a cell not regarded as aberrant or cancerous and may be considered an “average” of normal cell types.

The “immunointeractive molecule” is any molecule having specificity and binding affinity for StarD10 or its antigenic parts or its homologues or derivatives. Although the preferred immunointeractive molecule is an immunoglobulin molecule, the present invention extends to other immunointeractive molecules such as antibody fragments, single chain antibodies, de-immunized including humanized antibodies and T-cell associated antigen-binding molecules (TABMs). Most preferably, the immunointeractive molecule is an antibody such as a polyclonal or monoclonal antibody. Most preferably, the antibody is a monoclonal antibody.

The immunointeractive molecule exhibits specificity for StarD10 or more particularly an antigenic determinant or epitope on StarD10. An antigenic determinant or epitope on StarD10 includes that part of the molecule to which an immune response is directed. The antigenic determinant or epitope may be a B-cell epitope or where appropriate a T-cell epitope. The term “antigenic part” includes an antigenic determinant or epitope.

As described above, an “expression product” may be an mRNA or cDNA and the amount of expression product provides an indicator of the level of StarD10 gene expression and provides, therefore, indirect evidence for the presence of StarD10 protein. Conveniently, pools of mRNA or cDNA are obtained or cell extracts comprising total mRNA obtained and genetic probes complementary to all or part of the StarD10 gene-specific mRNA or cDNA. Binding of probes may then be quantified or semi-quantified.

Reference to a “level” of StarD10 includes an amount quantitatively, semi-quantitatively or qualitatively determined.

In accordance with the present invention, it is proposed that cells associated with a cancer and in particular mammary cancer cells produce elevated levels of StarD10. The quantitative or qualitative detection of levels of StarD10 or expression products of genetic material encoding StarD10 provides, therefore, an indicator that the cell is aberrant and is associated with cancer or has a propensity to develop into a cancer.

Accordingly, another aspect of the present invention contemplates a method for diagnosing the presence of cancer or cancer-like growth in a subject, said method comprising contacting cells or cell extracts from said subject or a biological sample from said subject with a StarD10-binding effective amount of an antibody having specificity for said StarD10 or an antigenic determinant or epitope thereon and then quantitatively or qualitatively determining the level of a StarD10-antibody complex wherein the presence of elevated levels of said complex compared to a normal cell is indicative of the presence of a cancer.

In a related embodiment, the present invention provides a method for diagnosing the presence of a cancer in a subject, said method comprising obtaining mRNA from cells of said subject or from a biological sample from said subject and optionally generating cDNA and contacting said mRNA or cDNA with a genetic probe capable of hybridizing to and/or amplifying all or part of a StarD10 nucleotide sequence encoding StarD10 or its complementary nucleotide sequence and then detecting the level of said mRNA or cDNA wherein the presence of elevated levels of said mRNA or cDNA compared to normal controls is indicative of the presence of cancer.

The use of antibodies and, in particular, monoclonal antibodies to detect StarD10 is the preferred method for use in this aspect of the present invention. Antibodies may be prepared by any of a number of means, as have been described above.

The present invention further contemplates the use of competitive drug screening assays, in which neutralizing antibodies capable of specifically binding the StarD10 polypeptide compete with a test compound for binding to the StarD10 polypeptide or fragments thereof. In this manner, the antibodies can be used to detect the presence of any peptide that shares one or more antigenic determinants of the StarD10 polypeptide. Such screening methods are not limited to assays employing only StarD10, but are also applicable to studying StarD10-protein complexes. The effect of drugs on the activity of this complex is analyzed.

Following identification of a substance that modulates or affects polypeptide activity, the substance may be investigated further. Furthermore, it may be manufactured and/or used in preparation, i.e. manufacture or formulation or a composition such as a medicament, pharmaceutical composition or drug. These may be administered to individuals in a method of treatment or prophylaxis.

The identification of StarD10 as a tumorigenesis-related molecule permits the generation of targeting agents to destroy or at least retard the growth of the cancer cells. In particular, the cancer targeting agents comprising StarD10-specific antibodies are fused, bound or otherwise associated with a cell growth inhibiting or killing agent. Such agents include but are not limited to cytocidal or cytostatic agents which act at the protein or corresponding mRNA or DNA levels. For example, the cell growth or killing agent maybe a nuclear tag or may be an agent which promotes induction of antagonists of StarD10 RNAi or RNA oligonucleotides.

Accordingly, another aspect of the present invention contemplates a method for the treatment of a patient having cancer, said method comprising administering to said human, a cancer cell growth inhibiting-effective amount of an antibody having specificity for human StarD10 protein, wherein said antibody is substantially non-immunogenic and further comprises a cell growth inhibiting or cell killing agent fused, bound or otherwise associated thereto.

The present invention further contemplates compositions comprising the cancer targeting agents of the present invention and one or more pharmaceutically acceptable carriers and/or diluents. The pharmaceutical forms suitable for injectable use include sterile aqueous solutions. It must be stable under the conditions of manufacture and storage and must be preserved against the contaminating action of microorganisms such as bacteria and fungi. The carrier can be a solvent or dilution medium comprising, for example, water, ethanol, polyol (for example, glycerol, propylene glycol and liquid polyethylene glycol, and the like), suitable mixtures thereof and vegetable oils. The proper fluidity can be maintained, for example, by the use of superfactants. The prevention of the action of microorganisms can be brought about by various anti-bacterial and anti-fungal agents, for example, parabens, chlorobutanol, phenol, sorbic acid, thirmerosal and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with the active ingredient and optionally other active ingredients as required, followed by filtered sterilization or other appropriate means of sterilization.

Pharmaceutically acceptable carriers and/or diluents include any and all solvents, dispersion media, coatings, anti-bacterial and anti-fungal agents, isotonic and absorption delaying agents and the like. The use of such media and agents for pharmaceutical active substances is well known in the art and except insofar as any conventional media or agent is incompatible with the active ingredient, their use in the therapeutic compositions is contemplated. Supplementary active ingredients can also be incorporated into the compositions.

The active agent is preferably administered in a therapeutically effective amount. The actual amount administered and the rate and time-course of administration will depend on the nature and severity of the condition being treated. Prescription of treatment, eg. decisions on dosage, timing, etc. is within the responsibility of general practitioners or specialists and typically takes account of the disorder to be treated, the condition of the individual patient, the site of delivery, the method of administration and other factors known to practitioners. Examples of techniques and protocols can be found in Remington's Pharmaceutical Sciences, 18^{th} Ed. (Mack Publishing Company, Easton, Pa., U.S.A., 1990).

Thus, the present invention extends to a pharmaceutical composition, medicament, drug or other composition comprising StarD10, a fragment thereof or a StarD10 antagonist or mimetic thereof, or a StarD10-specific antibody, associated with a cell growth inhibiting or killing agent, a method comprising administration of such a composition, a method comprising administration of such a composition to a patient, eg. for treatment (which may include preventative treatment) of cancer, use of such a composition in the manufacture of a composition for administration, eg. for treatment of cancer, and a method of making a pharmaceutical composition comprising admixing such a substance with a pharmaceutically acceptable excipient, vehicle or carrier, and optionally other ingredients.

A substance identified may be peptide or non-peptide in nature. Non-peptide “small molecules” are often preferred for many in vivo pharmaceutical uses. Accordingly, a mimetic or mimic of the substance (particularly if a peptide) may be designed for pharmaceutical use. The designing of mimetics to a known pharmaceutically active compound is a known approach to the development of a pharmaceutical based on a “lead” compound. This might be desirable where the active compound is difficult or expensive to synthesize or where it is unsuitable for a particular method of administration, eg. peptides are unsuitable active agents for oral compositions as they tend to he quickly degraded by proteases in the alimentary canal. Mimetic design, synthesis and testing are generally used to avoid randomly screening large numbers of molecules for a target property.

There are several steps commonly taken in the design of a mimetic from a compound having a given target property. First, the particular parts of the compound that are critical and/or important in determining the target property are determined. In the case of a peptide, this can be done by systematically varying the amino acid residues in the peptide, eg. by substituting each residue in turn. Alanine scans of peptide are commonly used to refine such peptide motifs. These parts or residues constituting the active region of the compound are known as its “pharmacophore”.

Once the pharmacophore has been found, its structure is modeled according to its physical properties, eg. stereochemistry, bonding, size and/or charge, using data from a range of sources, eg. spectroscopic techniques, x-ray diffraction data and NMR. Computational analysis, similarity mapping (which models the charge and/or volume of a pharmacophore, rather than the bonding between atoms) and other techniques can be used in this modeling process.

In a variant of this approach, the three-dimensional structure of the ligand and its binding partner are modeled. This can be especially useful where the ligand and/or binding partner change conformation on binding, allowing the model to take account of this in the design of the mimetic.

A template molecule is then selected, onto which chemical groups that mimic the pharmacophore can be grafted. The template molecule and the chemical groups grafted onto it can conveniently be selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property, or to what extent they exhibit it. Further optimization or modification can then be carried out to arrive at one or more final mimetics for in vivo or clinical testing.

The goal of rational drug design is to produce structural analogs of biologically active polypeptides of interest, or of small molecules with which they interact (eg. agonists, antagonists, inhibitors or enhancers), in order to fashion drugs which are, for example, more active or stable forms of the polypeptide, or which, eg. enhance or interfere with the function of a polypeptide in vivo. See, eg. Hodgson (Bio/Technology 9: 19-21, 1991). In one approach, one first determines the three-dimensional structure of a protein of interest (eg. StarD10) or, for example, of a StarD10 substrate or StarD10-ligand complex, by x-ray crystallography, by computer modeling or most typically, by a combination of approaches. Useful information regarding the structure of a polypeptide may also be gained by modeling based on the structure of homologous proteins. An example of rational drug design is the development of HIV protease inhibitors (Erickson et al., Science 249: 527-533, 1990). In addition, peptides (eg. StarD10) are analyzed by an alanine scan (Wells, 1991). In this technique, an amino acid residue is replaced by Ala and its effect on the peptide's activity is determined. Each of the amino acid residues of the peptide is analyzed in this manner to determine the important regions of the peptide.

It is also possible to isolate a target-specific antibody, selected by a functional assay and then to solve its crystal structure. In principle, this approach yields a pharmacophore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced banks of peptides. Selected peptides would then act as the pharmacophore.

Thus, one may design drugs which, for example, interfere with StarD10 activity or stability or which act as inhibitors or antagonists of StarD10 phosphoprotein, or which interact with StarD10 phosphoprotein or interfere with the phosphorylation of StarD10 phosphoprotein in such a way as to prevent or diminish its activity. By virtue of the availability of cloned StarD10 sequences, sufficient amounts of the StarD10 polypeptide may be made available to perform such analytical studies as x-ray crystallography. In addition, the knowledge of the StarD10 protein sequence provided herein will guide those employing computer modeling techniques in place of, or in addition to x-ray crystallography.

According to the present invention, a method is also provided for reducing or down-regulating StarD10 function in a cell that carries an active StarD10 gene. Removing such a function should suppress neoplastic growth in the recipient cells. The wild-type StarD10 gene or a part of the gene may be introduced into the cell in a vector such that the gene remains extra-chromosomal. In such a situation, the cell will express the gene from the extra-chromosomal location. Alternatively, it may be integrated into the nuclear genome of the cell. If a StarD10 gene or a nucleic acid sequence comprising a portion of the StarD10 gene, or a nucleic acid sequence complementary to a StarD10 gene sequence or portion thereof, is introduced and expressed in a cell, it may lead to the down-regulation of the endogenous StarD10 gene present in the cell.

The term “down-regulating” is used in its most generic sense and includes any and all means by which the expression of a gene is reduced, diminished or eliminated, whether at the transcriptional or the post-transcriptional level.

Vectors for introduction of genes both for recombination and for extra-chromosomal maintenance are known in the art and any suitable vector may be used. Methods for introducing DNA into cells such as electroporation, calcium phosphate co-precipitation and viral transduction are known in the art and the choice of method is within the competence of the practitioner.

As generally discussed above, the StarD10 gene or fragment, where applicable, may be employed in gene therapy methods in order to down-regulate gene expression and thereby decrease the amount of the expression products of such genes in cancer cells. Such gene therapy is particularly appropriate for use in both cancerous and pre-cancerous cells, in which the level of StarD10 polypeptide is absent or diminished compared to normal cells.

One for of gene therapy is carried out according to generally accepted methods, for example, as described by Friedman (In: Therapy for Genetic Disease, T. Friedman, Ed., Oxford University Press, pp. 105-121, 1991) or Culver (Gene Therapy. A Primer for Physicians, 2^{nd} Ed., Mary Ann Liebert, 1996). Cells from a patient's tumor would be first analyzed by the diagnostic methods described above, to ascertain the production of StarD10 polypeptide in the tumor cells. A virus or plasmid vector, containing a copy of the StarD10 gene linked to expression control elements is prepared. The vector may be capable of replicating inside the tumor cells. Suitable vectors are known, such as disclosed in U.S. Pat. No. 5,252,479, International Patent Publication No. WO 93/07282 and U.S. Pat. No. 5,691,198. The vector is then injected into the patient, either locally at the site of the tumor or systemically (in order to reach any tumor cells that may have metastasized to other sites). If the transfected gene is not permanently incorporated into the genome of each of the targeted tumor cells, the treatment may have to be repeated periodically. The present invention further contemplates using sense sequences of StarD10 to induce co-suppression or other forms of post transcriptional gene silencing as well as any mechanism involving RNA₂ and/or epigenetic mechanisms to silence gene expression.

Gene transfer systems known in the art may be useful in the practice of the gene therapy methods of the present invention. These include viral and non-viral transfer methods. A number of viruses have been used as gene transfer vectors or as the basis for preparing gene transfer vectors, including papovaviruses (eg. SV40, Madzak et al., J. Gen. Virol. 73: 1533-1536, 1992), adenovirus (Berkner, Curr. Top. Microbiol. Immunol. 158: 39-66, 1992; Berkner et al., BioTechniques 6; 616-629, 1988; Gorziglia and Kapikian, J. Virol. 66: 4407-4412, 1992; Quantin et al., Proc. Natl. Acad. Sci. USA 89: 2581-2584, 1992; Rosenfeld et al., Cell 68: 143-155, 1992; Wilkinson et al., Nucleic Acids Res. 20: 2233-2239, 1992; Stratford-Perricaudet et al., Hum. Gene Ther. 1: 241-256, 1990; Schneider et al., Nature Genetics 18: 180-183, 1998), vaccinia virus (Moss, Curr. Top. Microbiol. Immunol. 158: 25-38, 1992; Moss, Proc. Natl. Acad. Sci. USA 93: 11341-11348, 1996), adeno-associated virus (Muzyczka, Curr. Top. Microbiol. Immunool. 158: 97-129, 1992; Ohi et al., Gene 89: 279-282, 1990; Russell and Hirata, Nature Genetics 18: 323-328, 1998), herpesviruses including HSV and EBV (Margolskee, Curr. Top., Microbiol. Immunol. 158: 67-95, 1992; Johnson et al., J. Virol. 66: 2952-2965, 1992; Fink et al., Hum. Gene Ther. 3: 11-19, 1992; Breakefield and Geller, Mol. Neurobiol. 1: 339-371, 1987; Freese et al., Biochem. Pharmacol. 40: 2189-2199, 1990; Fink et al., Ann. Rev. Neurosci. 19: 265-287, 1996), lentiviruses (Naldini et al., Science 272: 263-267, 1996), Sindbis and Semliki Forest virus (Berglund et al., Biotechnology 11: 916-920, 1993) and retroviruses of avian (Bandyopadhyay and Temin, Mol. Cell. Biol. 4: 749-754, 1984; Petropoulos et al., J. Viol. 66: 3391-3397, 1992], murine [Miller, Curr. Top. Microbiol. Immunol. 158: 1-24, 1992; Miller et al., Mol. Cell. Biol. 5: 431-437, 1985; Sorge et al., Mol. Cell. Biol. 4: 1730-1737, 1984; Mann and Baltimore, J. Virol. 54: 401-407, 1985; Miller et al., J. Virol. 62: 4337-4345, 1988] and human [Shimada et al., J. Clin. Invest. 88: 1043-1047, 1991; Helseth et al., J. Virool. 64: 2416-2420, 1990; Page et al., J. Virol. 64: 5270-5276, 1990; Buchschacher and Panganiban, J. Virol. 66: 2731-2739, 1982] origin. Most human gene therapy protocols have been based on disabled murine retroviruses although adenovirus and adeno-associated virus are also being used.

Non-viral gene transfer methods are known in the art such as chemical techniques including calcium phosphate co-precipitation, mechanical techniques, for example, micro-injection, membrane fusion-mediated transfer via liposomes and direct DNA uptake and receptor-mediated DNA transfer. Viral-mediated gene transfer can be combined with direct in vivo gene transfer using liposome delivery, allowing one to direct the viral vectors to the tumor cells and not into the surrounding non-dividing cells. Alternatively, the retroviral vector producer cell line can be injected into tumors. Injection of producer cells would then provide a continuous source of vector particles.

In an approach which combines biological and physical gene transfer methods, plasmid DNA of any size is combined with a polylysine-conjugated antibody specific to the adenovirus hexon protein and the resulting complex is bound to an adenovirus vector. The trimolecular complex is then used to infect cells. The adenovirus vector permits efficient binding, internalization and degradation of the endosome before the coupled DNA is damaged. For other techniques for the delivery of adenovirus based vectors, see U.S. Pat. No. 5,691,198.

Liposome/DNA complexes have been shown to be capable of mediating direct in vivo gene transfer. While in standard liposome preparations the gene transfer process is non-specific, localized in vivo uptake and expression have been reported in tumor deposits, for example, following direct in situ administration.

Expression vectors in the context of gene therapy are meant to include those constructs containing sequences sufficient to express a polynucleotide that has been cloned therein. In viral expression vectors, the construct contains viral sequences sufficient to support packaging of the construct. If the polynucleotide encodes StarD10, expression will produce StarD10. If the polynucleotide encodes a sense or antisense polynucleotide or a ribozyme or DNAzyme, expression will produce the sense or antisense polynucleotide or ribozyme or DNAzyme. Thus, in this context, expression does not require that a protein product be synthesized. In addition to the polynucleotide cloned into the expression vector, the vector also contains a promoter functional in eukaryotic cells. The cloned polynucleotide sequence is under control of this promoter. Suitable eukaryotic promoters include those described above. The expression vector may also include sequences, such as selectable markers and other sequences described herein.

The therapy is as follows: patients who carry a StarD10 gene are treated with a gene delivery vehicle such that some or all of their precursor cells receive at least one additional copy of said gene, in sense or antisense and/or combinations thereof; so as to effect interference with the expression of the endogenous StarD10 sequence. In this step, the treated individuals thereby have reduced risk of development of the cancer to the extent that the expression of the endogenous StarD10 gene has been prevented and/or diminished by the introduction of the additional copies of a part or all of a nucleotide sequence encoding StarD10.

Accordingly, in this embodiment, the present invention contemplates genetic constructs such as comprising antisense, sense and ribozyme constructs or RNAi or RNA oligonucleotides. Such genetic compositions are also referred to as DNA vaccination compositions and are specifically directed to modulating expression, at the transcriptional or translational levels, of nucleotide sequences encoding a StarD10 protein.

Accordingly, another aspect of the present invention contemplates a method for the treatment of a patient having cancer, said method comprising administering to said patient, a genetic composition comprising a genetic construct which down-regulates expression of a StarD10 gene encoding StarD10.

More particularly, the present invention contemplates a method for the treatment of a patient having cancer, said method comprising administering to said patient, a genetic composition comprising a genetic construct comprising a nucleotide sequence substantially as set forth in SEQ ID NO:4 or a fragment thereof or a nucleotide sequence having at least about 40% similarity to SEQ ID NO:4 after optimal alignment or a nucleotide sequence capable of hybridizing to SEQ ID NO:4 its complementary form under low stringency conditions.

Reference herein to “expression” includes down-regulating the steps of transcription, translation or both. Particularly preferred genetic constructs are antisense or sense constructs to StarD10, which induce co-suppression of the StarD10 gene or induce RNAi-mediated down-regulation of StarD10 mRNA transcript.

In another aspect of the present invention, cells and animals which carry a StarD10 nucleic acid sequence can be used as model systems to study and test for substances which have potential as therapeutic agents. Cells may be isolated, for example, from individuals expressing a StarD10 gene. Alternatively, a cell line may be engineered to carry the StarD10 gene or, in an alternative approach, an aberration in the StarD10 sequence. Mutating or deleting the StarD10 gene or an allele thereof has the ability to reduce or prevent its expression. The term “mutating”, and related terms such as “mutate”, “mutation” and “mutated”, are used in their most generic sense and includes methylation of all or part of the nucleotide sequences, and in particular cytosine or guanine residues, or other events which induces gene silencing.

The term “aberration” encompasses all forms of mutations including deletions, insertions, point mutations and substitutions in the coding and non-coding regions of StarD10. It also includes changes in methylation patterns of StarD10 or of an allele of StarD10. Deletions may be of the entire gene or only a portion of the gene. Point mutations may result in stop codons, frame-shift mutations or amino acid substitutions. Somatic mutations are those occurring only in certain tissues, eg. in the tumor tissue and are not inherited in the germ-line. Germ-line mutations can be found in any of a body's tissues and are inherited. A StarD10 allele, which is not deleted (eg. that found on the sister chromosome to a chromosome carrying a StarD10 deletion), can be screened for other mutations such as insertions, small deletions, point mutations and changes in methylation pattern. Mutations leading to non-functional gene products may also lead to decreased prevalence of a cancerous state. Point mutational events may occur in regulator regions, such as in the promoter of the gene or in an intron of the gene, leading to loss or diminution of expression of the mRNA. Point mutations may also abolish proper RNA processing leading to loss of expression of the StarD10 gene product or a decrease in mRNA stability or translation efficiency. Point and other mutations may also affect proper RNA processing such as intron splicing.

After a test substance is applied to the cells, the neoplastically-transformed phenotype of the cell is determined. Any trait of neoplastically-transformed cells can be assessed, including anchorage-independent growth, tumorigenicity in nude mice, invasiveness of cells and growth factor dependence. Assays for each of these traits are known in the art.

Animals for testing therapeutic agents can be selected after mutagenesis of whole animals or after treatment of germ-line cells or zygotes. Such treatments include insertion of StarD10 genes, usually from a second animal species, and may also include insertion of disrupted homologous genes. Alternatively, an endogenous StarD10 gene of an animal may be disrupted by insertion or deletion mutation or other genetic alterations using conventional techniques. After test substances have been administered to the animals, the growth of tumors must be assessed. If the test substance prevents or suppresses the growth of tumors, then the test substance is a candidate therapeutic agent for the treatment of the cancers identified herein. These animal models provide an extremely important testing vehicle for potential therapeutic products.

The identification of the association between the presence of StarD10 phosphoprotein and cancer permits the early pre-symptomatic screening of individuals to identify those at risk for developing cancer. To identify such individuals, they are screened for the presence of StarD10, or for StarD10 sequences or the presence of nucleic acid sequence differences from the normal gene using any suitable technique. Suitable techniques include but are not limited to, one of the methods hereinbefore described, i.e. fluorescent in situ hybridization (FISH), direct DNA sequencing, PFGE analysis, Southern blot analysis, single-stranded conformation analysis (SSCP), linkage analysis, RNase protection assay, allele specific oligonucleotide (ASO), dot blot analysis and PCR-SSCP analysis, or DNA/RNA microchip technology.

An alternative strategy is a large scale immunoaffinity purification of StarD10-biomolecular complexes. The partners in the purified complex can then be eluted off and analysed.

Two-hybrid screening is particularly useful in identifying other members of a biochemical or genetic pathway associated with StarD10. Two-hybrid screening conveniently uses Saccharomyces cerevisiae and Saccharomyces pombe. StarD10 interactions and screens for inhibitors can be carried out using the yeast two-hybrid system, which takes advantage of transcriptional factors that are composed of two physically separable, functional domains (Phizicky and Fields, Microbiol. Rev. 59(1): 94-123, 1994). The most commonly used is the yeast GAL4 transcriptional activator consisting of a DNA binding domain and a transcriptional activation domain. Two different cloning vectors are used to generate separate fusions of the GAL4 domains to genes encoding potential binding proteins. The fusion proteins are co-expressed, targeted to the nucleus and if interactions occur, activation of a reporter gene (eg. lacZ) produces a detectable phenotype. In the present case, for example, S. cerevisiae is co-transformed with a library or vector expressing a cDNA GAL4 activation domain fusion and a vector expressing a StarD10-GAL4 binding domain fusion. If lacZ is used as the reporter gene, co-expression of the fusion proteins will produce a blue color. Small molecules or other candidate compounds which interact with StarD10 will result in loss of color of the cells. This system can be used to screen for small molecules that inhibit the StarD10 interaction and, hence, protect the yeast against cell death and to determine the residues in StarD10 which are involved in their interaction. For example, reference may be made to the yeast two-hybrid systems as disclosed by Munder et al. (Appl. Microbiol. Biotechnol. 52(3): 311-320, 1999) and Young et al., Nat. Biotechnol. 16(10): 946-950, 1998). Molecules thus identified by this system are then re-tested in animal cells.

The present invention is further described by the following non-limiting Examples.

### EXAMPLE 1

### Reagents and Mice

Reagents

Antibodies used were as follows: affinity purified rabbit polyclonal anti-phospho-FKHR (S256) antibody (New England Biolabs); mouse monoclonal anti-α-tubulin antibody (clone B-5-1-2; Sigma); mouse monoclonal anti-ErbB2 antibody (Ab-10; Neomarkers); mouse monoclonal anti-Neu antibody (Ab-3; Calbiochem); mouse monoclonal anti-Flag antibody (Sigma); anti-Pctp1 polyclonal rabbit serum. Recombinant human EGF and insulin were purchased from Sigma.

Mice

Mouse mammary tumor virus (MMTV)-Neu mice have been previously described (Guy et al. (2000), supra. Nulliparous female mice were aged for 8-12 months in standard animal facilities and were sacrificed when tumors developed.

### EXAMPLE 2

### Preparation of Protein Lysates

To explore the potential inactivation of the FKHR transcription factor by ErbB receptor signaling, protein lysates prepared from mammary tumors derived from mouse mammary tumor virus (MMTV)-Neu transgenic mice. Whole cell extracts were obtained by solubilizing cells in 1% NEB (50 mM Tris, pH 7.5, 150 mM NaCl, 1% NP40, 1 mM sodium orthovanadate, 10 mM sodium fluoride, 20 mM β-glycerophosphate plus complete protease inhibitors (Roche)). Lysates were clarified by centrifugation at 16,000×g for 10 min. Protein lysates of tissues were obtained by grinding the tissue to a fine powder under liquid nitrogen and subsequent solubilization in 1% TEB (NEB with substitution of NP40 with Triton X-100). Lysates were homogenized by passing through a 21-gauge needle prior to clarification by centrifugation.

For cytosolic protein extraction, cells were lysed in hypotonic buffer (10 mM Hepes, pH 7.9, 133 mM sorbitol, 0.5 mM sodium fluoride, 0.5 mM β-glycerophosphate plus complete protease inhibitors (Roche)), left to swell on ice for 10 min, homogenized by Douncing and then centrifuged at 800×g for 10 min. The pellet was washed with hypotonic buffer and the supernatants were combined to yield the cytosolic fraction.

### EXAMPLE 3

### A Tumor-Specific 35 kDa Protein Cross-Reacts with a Phosphospecific FKHR Antibody

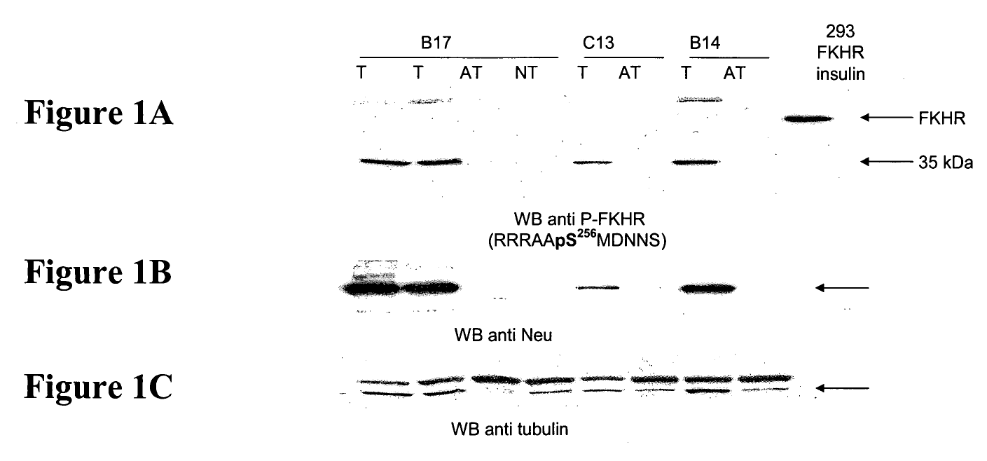

Mammary tumor lysates were analyzed by Western blotting, using an antibody that specifically recognizes FKHR phosphorylated on serine 256 (FIG. 1A).

The proteins were blotted onto polyvinylidine difluoride membranes (Millipore). After blocking with 20% v/v horse serum (Hunter) in PBS containing 0.1% v/v Tween-20, filters were probed with specific antibodies. Proteins were visualized with peroxidase-coupled secondary antibody using the ECL detection system (Amersham). Stripping of membranes was performed in SDS buffer (62.5 mM Tris, pH 6.8, 2% v/v SDS, 100 mM β-mercaptoethanol) for 30 min at 60° C. Membranes were then re-probed with the indicated antibodies.

No signal corresponding to FKHR was detected in tumor or adjacent mammary tissue, reflecting the low level of FKHR expression. Interestingly, the phosphospecific FKHR antibody strongly cross-reacted with a 35 kD protein that was present in tumor but not in adjacent or normal mammary tissue, as shown in FIG. 1. Western blotting with an anti-Neu antibody confirmed over-expression Neu in these tumors.

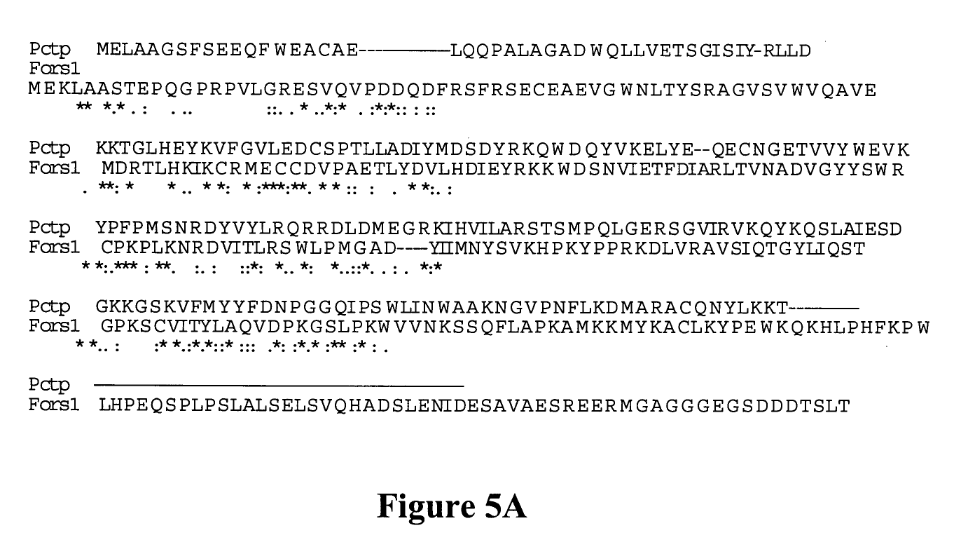

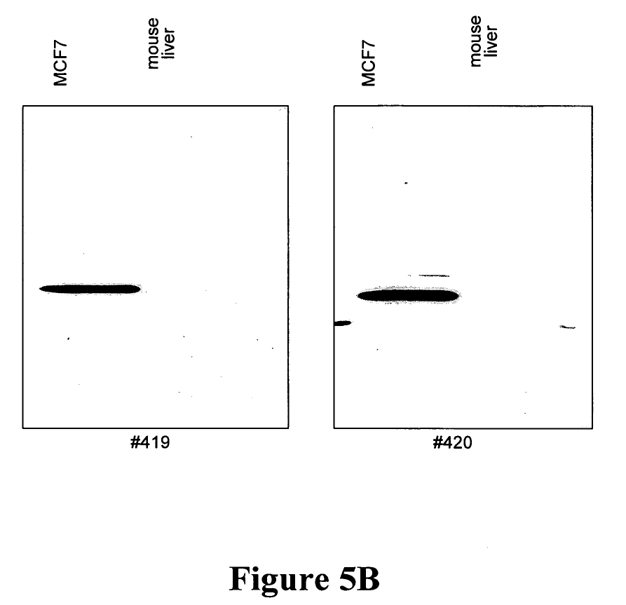

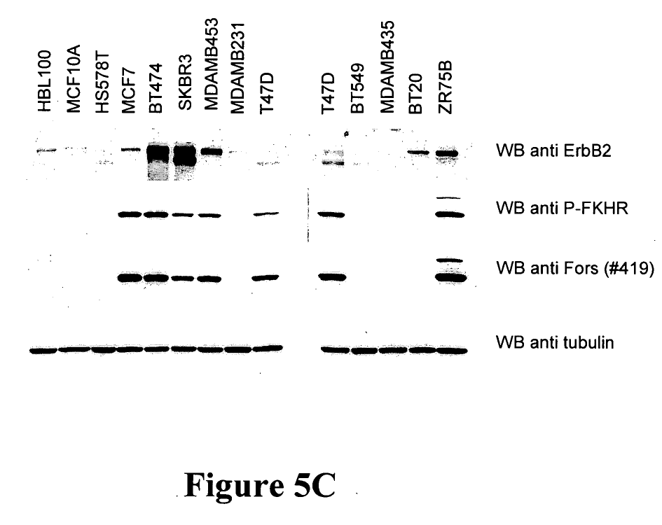

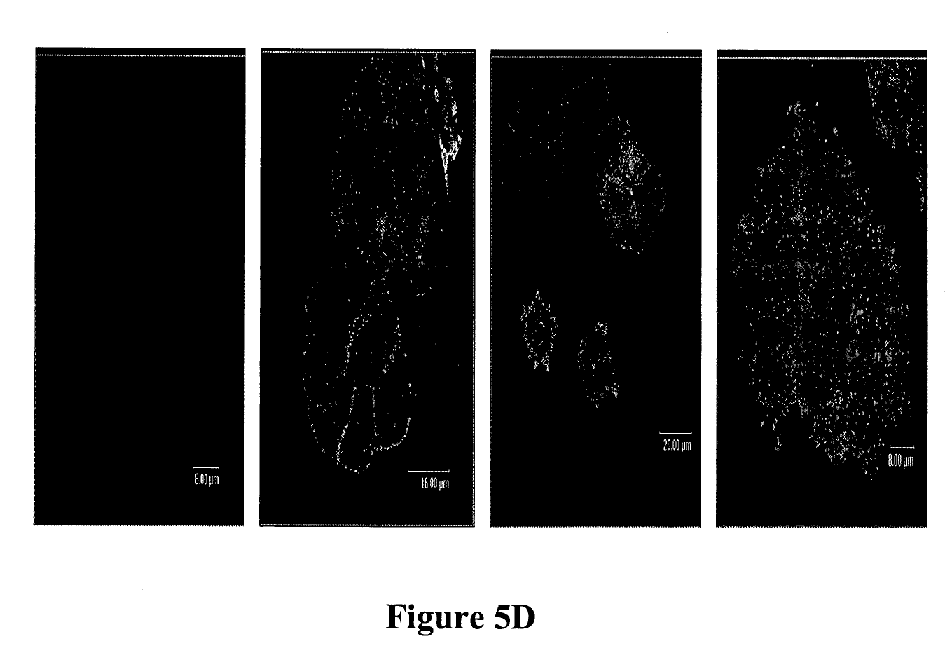

Further analysis of several breast epithelial cell lines revealed that the 35 kD protein was absent in the immortalized breast epithelial cell lines HBL100 and 184 but could be detected in the majority of transformed cell lines (refer to Example 9, below, and to FIG. 5C).

Since no FoxO family members corresponding to this molecular weight had been described, it was hypothesized that the 35 kD protein might represent a novel FKHR-related protein. This protein was likely to be phosphorylated on a serine residue lying in a conserved sequence context, given the specificity of the FKHR-specific antibody.

### EXAMPLE 4

### Purification of the 35 kD Protein

In order to identify the protein cross-reacting with the phospho-FKHR antibody, an immunoprecipitation with this antibody using SKBR3 cell lysate was performed.

For immunoprecipitations, equal amounts of protein were incubated with specific antibodies for 2 h on ice. Immune complexes were collected with protein G-Sepharose (Pharmacia) and washed three times with 1% NEB. For phosphorylation analysis, immune complexes were further washed with 10 mM Tris, pH 8, 100 mM NaCl, before being incubated in the same buffer with 5 units of alkaline phosphatase (Roche) for 45 min at 37° C. Precipitated proteins were released by boiling in sample buffer and were subjected to sodium dodecyl sulfate (SDS)-polyacrylamide gel electrophoresis (PAGE) using 4-20% gradient gels (Novex).

While the antibody readily detected the 35 kDa protein by Western blotting, it failed to immunoprecipitate the protein. A multi-step biochemical purification strategy was therefore devised, based on parameters such as isoelectric focusing point, hydrophobicity and molecular weight of the protein. Since the 35 kD protein was demonstrated to be cytosolic, protein lysates from SKBR3 cells were prepared by hypotonic lysis using conditions that are compatible with isoelectric focusing (IEF). An analytical sample of SKBR3 cytosol was initially analyzed by free flow electrophoresis (FFE), a liquid based isoelectric focusing method.

FFE was essentially performed as described in Hoffmann et al., Proteomics 1: 807-818, 2001, using the Octopus apparatus. The IEF running buffer was aqueous 0.2% w/v HPMC (hydroxypropyl methyl cellulose) and 0.2% w/v carrier ampholytes (Servalyte pH 3-10). Electrode solutions were 100 mM H₃PO₄ (anode) and 50 mM NaOH (cathode); the counter flow solution (0.7 l/min) was 0.2% HPMC containing 0.02 M L-arginine and 0.02 M L-lysine. For preparative separations, the sample was diluted to a final concentration of approximately 0.25 mg protein per ml. Electrophoresis was performed at 4° C. with a flow-rate of 1.4 ml/min and a constant voltage of 1250 V.

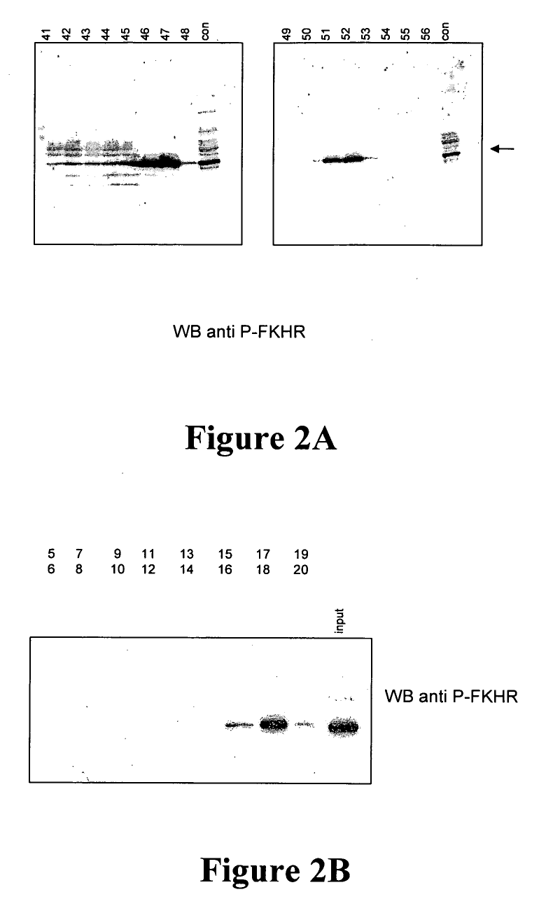

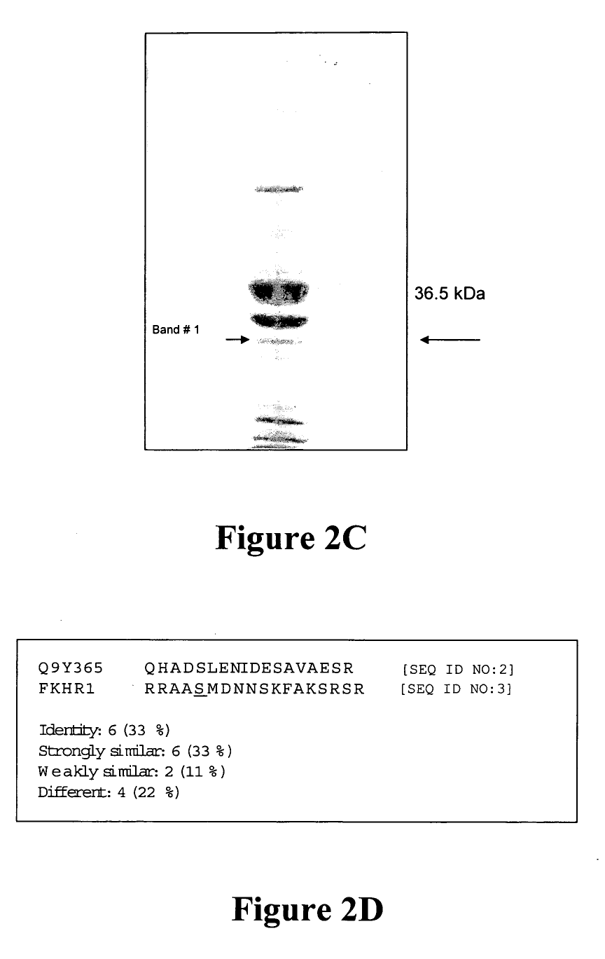

This continuous mode of isoelectric focusing is especially advantageous for protein purification when large volumes of cell lysate are required. As shown in FIG. 2A, Western blotting of individual fractions using the phospho-FKHR antibody revealed the presence of two protein bands with distinct isoelectric focusing points, corresponding to pH 5.6 and pH 6. This shift may reflect differential phosphorylation.

To test whether the 35 kD protein could be effectively separated by reverse phase HPLC, SKBR3 cytosol was subjected to RP-HPLC. The 35 kD protein was found to elute in specific fractions, as shown in FIG. 2B.

For preparative purification of the 35 kD protein, 275 mg of cytosolic protein was applied to FFE followed by RP-HPLC. Fractions containing the 35 kD protein were identified by Western blotting using the phospho-FKHR antibody, then pooled (7 mg) and subjected to RP-HPLC. HPLC fractions containing the 35 kD protein were pooled and precipitated by addition of 0.5% w/v deoxycholate and 15% v/v TCA plus two volumes of acetone at −20° C. After centrifugation at 16,000×g for 15 min, the protein pellet was washed with acetone, briefly left to dry and resuspended in sample buffer. A large 10% w/v gel was poured according to Laemmli and electrophoresis was performed at 150 V for 6 hours. The gel was fixed and stained with Coomassie Phast-gel Blue R (Pharmacia, UK) (FIG. 2C).

The bands migrating at approximately 35 kD were excised, digested in situ with trypsin and analyzed by tandem mass spectrometry. All peptides from band #1 matched an entry in the Tremb1 database, Q9Y365, a predicted human protein based on a comparative screen of the C. elegans proteome with human est nucleotide databases (Lai et al., Genome Research 10: 703-713, 2000). Alignment of this sequence with the FKHR protein failed to identify a forkhead domain but instead identified a region in Q9Y365 that was highly similar to the sequence surrounding serine 256 to which the phospho-FKHR antibody had been raised (FIG. 2D). Most importantly, this sequence in Q9Y365 contained a serine that (i) aligned with S256 in FKHR and (ii) obtained a high score in the Netphos phosphorylation prediction program. It, therefore, appeared likely that this protein, when phosphorylated on this serine residue, would cross-react with the FKHR-specific antibody.

### EXAMPLE 5

### Cloning and Expression Analysis of the 35 kD Protein, StarD10

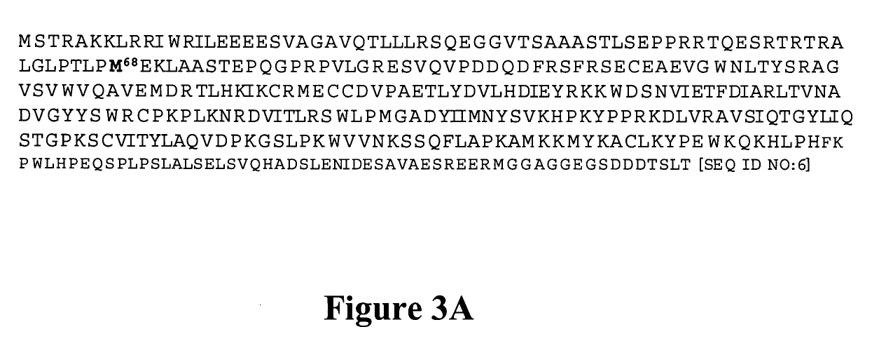

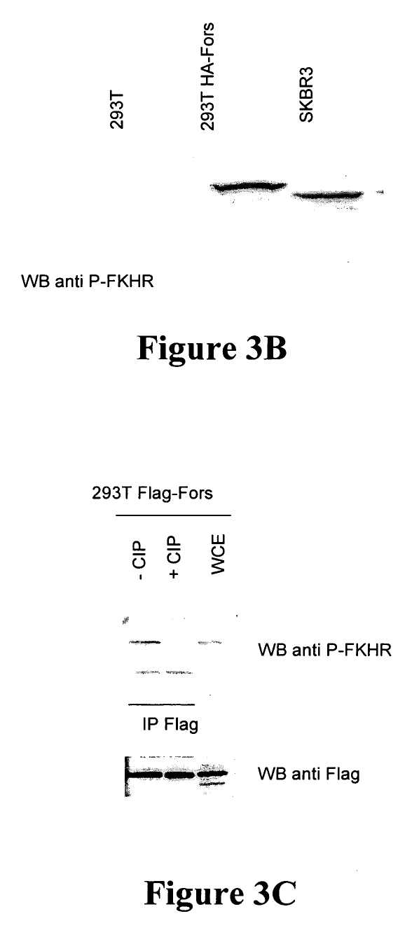

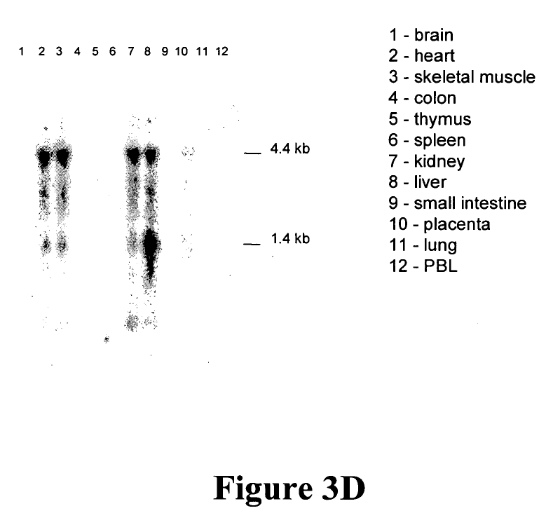

Sequence analysis of Q9Y365 predicted a molecular weight of 40 kD and a pI of 8.55, higher than the experimentally observed values of the 35 kD protein. No sequence coverage of the first 71 amino acids was obtained, suggesting that translation was initiated from the methionine at position 68, giving rise to a protein of 291 amino acids with a theoretical mass of 33 kD and a pI of 6.7. These calculations do not take into account the presence of phosphorylation sites, which would render the protein more acidic (FIG. 3A) and would decrease the electrophoretic mobility in SDS-PAGE.

Full-length cDNA encoding a 33 kD protein product was generated by RT-PCR using RNA derived from SKBR3 cells. Total RNA was extracted from SKBR3 cells using RNAzol (Tel-Test). First strand cDNA synthesis was performed and StarD10 cDNA was amplified by PCR using primers based on the CGI-52 sequence (Accession No. AF151810). StarD10 cDNA was cloned into HA-pcDNA3.0, Flag-pEFrPGKpuro and pBabePuro for expression in mammalian cells. An HA or Flag tag was incorporated at the N-terminus within the pcDNA3.0 or pEF-1αrev expression vectors, respectively, to facilitate recognition.

Remarkably, transient expression of the HA-tagged expression plasmid in 293T cells gave rise to a protein that was detected by the phospho-FKHR antibody (FIG. 3B). The slight difference in size compared to the endogenous protein is most likely due to the presence of the tag. Recognition of this protein was phosphorylation-dependent, since treatment of immunoprecipitated Flag-tagged protein with alkaline phosphatase abrogated the cross-reactivity. This suggests that serine 259 in the 35 kD protein is indeed phosphorylated. This protein was, therefore, termed StarD10 (forkhead related serine).

Although the existence of a long human StarD10 protein as predicted by Lai et al. (2000) supra cannot be ruled out, Pctp2, the mouse homolog of StarD10, was cloned as a 291 amino acid protein from a mouse testis library. The mouse sequence does not indicate the presence of larger protein since there is no open reading frame encoding an amino-terminal extension, distinct from that observed in the human sequence.

### EXAMPLE 6

### Northern Analysis of StarD10 mRNA

Poly(A)⁺ RNA was isolated from breast epithelial cell lines and Northern analysis was performed as described (Visvader, EMBO J. 11: 4557-4564, 1991). Briefly, 4 μg of poly(A)⁺ RNA was fractionated on 1% w/v agarose-formaldehyde gel, transferred to Hybond N⁺ (Amersham Pharmacia Biotech), and hybridized with the following cDNA probes: StarD10 cDNA, encompassing the entire 876 bp coding region or residues 769-876, ErbB2 cDNA (residues 1-228), EGFR cDNA (residues 1-293) or GAPDH cDNA. Human multiple poly(A)⁺ RNA tissue Northern blots (Clontech Laboratories, Inc.) were hybridized with a StarD10 cDNA probe.

Northern analysis of StarD10 mRNA from a variety of human organs revealed high levels of a 1.4 kb transcript in liver, heart, skeletal muscle and kidney. While a faint signal was detected in placenta, organs such as brain, colon, thymus, spleen, small intestine, lung and PBL cells lacked StarD10 mRNA.

### EXAMPLE 7

### Co-Expression of StarD10 and ErbB2 Occurs at High Levels in Breast Cancer Cell Lines

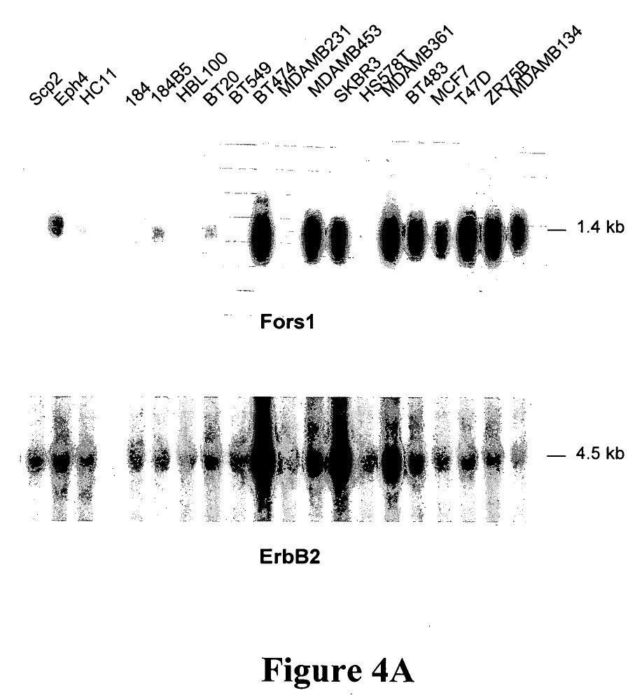

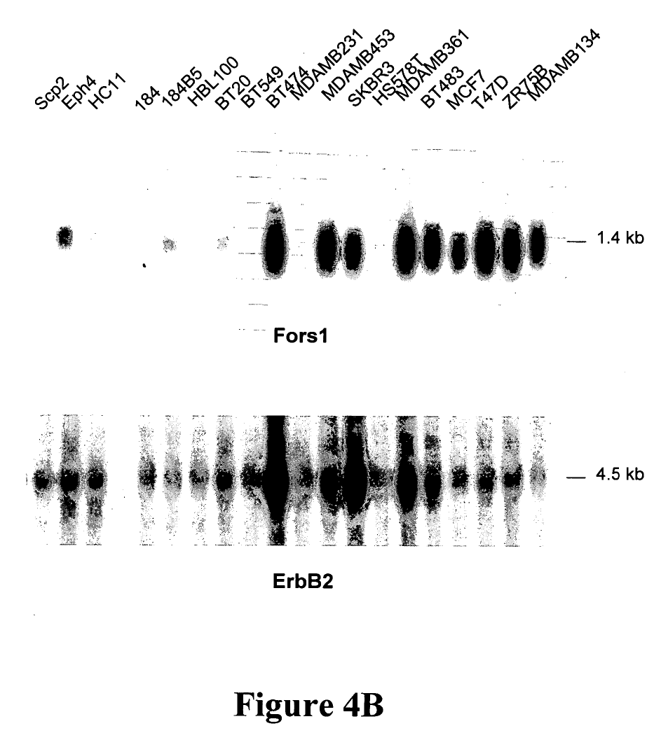

StarD10 levels were originally found to be abundant in tumors from Neu transgenic mice and in breast carcinoma cell lines using an antibody that recognizes the protein in a phosphorylation-dependent manner. To determine whether StarD10 up-regulation was occurring at the RNA level and whether it correlated with over-expression of ErbB2, Northern analysis was performed, as described in Example 6, above, on a panel of breast epithelial cell lines (FIG. 4A).

High levels of StarD10 were found in the cell lines that were previously shown to express phosphorylated StarD10 protein, indicating over-expression at the transcriptional level. StarD10 expression was barely detectable in both human and mouse immortalized cell lines (184, HBL100, Scp2, Eph4 and HCl1). Interestingly, all cell lines that over-expressed ErbB2 mRNA were established to have higher StarD10 levels. StarD10 over-expression, however, was also noted in cell lines without a concomitant increase in ErbB2 expression.

An expression survey of human colon carcinoma cell lines suggested that over-expression of StarD10 might also be a feature of this tumor type (FIG. 4B). Several cell lines expressed StarD10 to a level that was comparable to that seen in SKBR3 cells. However, there was no correlation between StarD10 and EGFR mRNA.

### EXAMPLE 8

### Culture of Cell Lines

Breast epithelial cell lines were maintained in RPMI containing 10% v/v fetal bovine serum (FBS) (CSL) and 1 μg/ml insulin with the exception of MCF10A cells that were grown in DME HAM supplemented with 10% μ FBS, 10 ng/ml EGF, 5 μg/ml insulin and 1 μM dexamethasone.

293T, NIH3T3 and HER14 (NIH3T3 transfected with the human EGFR) and Bosc cell lines were grown in DME medium containing 10% v/v FBS. NIH3T3 StarD10 and HER14 StarD10 were generated by stable introduction of a pBabe retrovirus encoding HA-tagged StarD10 using Bosc packaging cells and selection with 1 μg/ml puromycin (Sigma).

For transient transfections, 293T cells were transfected with Fugene reagent (Roche) according to the manufacturer's instructions.

### EXAMPLE 9

### Generation of StarD10-Specific Antibodies

Database searches identified the presence of a Start domain in StarD10 (amino acid 21-226), a phospholipid binding domain first noted in the steroidogenic acute regulatory protein Star. The Start domain of StarD10 is 28% identical and 49% similar to that of human Pctp (phosphatidycholine transfer protein), a 214 amino acid protein which consists almost entirely of the Start domain. Unlike Pctp, StarD10 has a 65 amino acid carboxyterminal extension (FIG. 5A).

To generate an antibody specific for StarD10, rabbits were immunized with a keyhole limpet hemocyanin (KLH; Sigma)-coupled peptide encompassing amino acids 265-277 at the C-terminus downstream of the Start domain. The peptide ESAVAESREERMG (SEQ ID NO:1) corresponding to amino acids 265-277 of StarD10 was selected, based on antigenicity and surface availability predictions and synthesized with an additional aminoterminal cysteine (Auspep) for coupling to KLH. In brief, KLH was cross-linked with N-Succinimidyl 3-(2-pyridyldithio)propionate (Pierce) and added to peptide that had been reduced and purified with SepPak columns (Waters). Two rabbits (#419 and 420) were injected subcutaneously with 200 μg peptide in complete adjuvants and boosted in 2 weekly intervals. Bleeds were tested by ELISA and Western blotting. Antibody purification from rabbit sera was performed with peptide that had been covalently coupled to Sulfolink coupling gel (Pierce) following the manufacturer's instructions. Elution was carried out with 100 mM glycine, pH 2.7 and antibody-containing fractions were pooled and dialyzed against PBS.

### EXAMPLE 10

### Localization of StarD10 in Breast Epithelial Cells

Upon affinity purification, both antibodies were found to specifically recognize human StarD10 protein (FIG. 5B). Immunoblotting of breast epithelial cell lines confirmed that over-expression of phosphorylated StarD10 protein correlates with ErbB2 status in many cases (FIG. 5C).

Immunofluorescence of MCF7 cells was undertaken, with a StarD10-specific antibody, using confocal microscopy. Cells were plated onto cover-slips in 6-well dishes 24 hours prior to immunostaining. Cells were fixed with 4% v/v paraformaldehyde in PBS for 10 min, permeabilized with PBS-T (PBS containing 0.2 Triton X-100) and then blocked (5% goat serum (Hunter) in PBS-T) for 30 min. Cover-slips were incubated for 1 hour with affinity purified StarD10-specific polyclonal antibody (#420) diluted in blocking solution. After washing with PBS-T, cover-slips were incubated with anti-rabbit secondary antibody conjugated to fluorescein isothiocyanate (The Jackson Laboratory). Cells were counterstained with 1 μg/ml Bis-benzimide (Sigma) and cover-slips were then mounted onto slides with fluorescent mounting medium (Dako Corp., Carpinteria, Calif.). Cells were examined using a Leica Corp. TCS-4D confocal microscope.

Results revealed punctate staining in the cytoplasm and some staining in the nucleus. Interestingly, StarD10 also appeared to be concentrated at the boundaries of cell clusters (FIG. 5B). Scansite sequence analysis of StarD10 suggests that the carboxyterminus may harbor a PDZ domain recognition motif. It will thus be interesting to determine the intracellular compartment to which StarD10 localizes and whether its localization is controlled by PDZ interactions.

### EXAMPLE 11

### StarD10 Cooperates with ErbB1 in Cellular Transformation

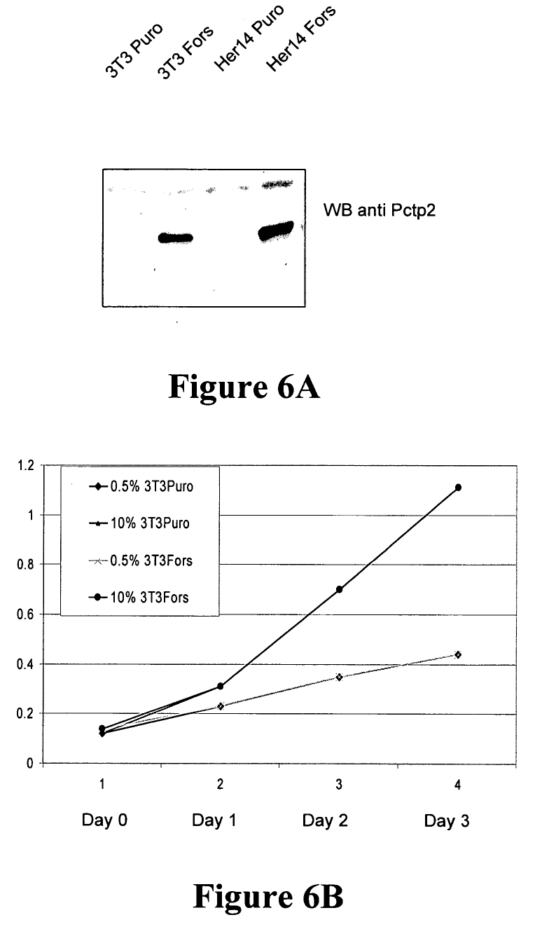

To investigate the effect of StarD10 over-expression on cell proliferation, NIH3T3 and HER14 cells stably over-expressing StarD10 were generated by retroviral infection. The HER14 line represents NIH3T3 cells stably expressing the EGFR. Parental NIH3T3 and HER14 cells lack StarD10, determined by Western blotting using an anti-Pctp2 antibody which recognizes both the mouse and human protein (FIG. 6A).

StarD10 over-expression did not appear to alter the morphology of either NIH3T3 or HER14 cells. Growth was assessed by MTS assays, carried out by plating 2000 cells per well (96 well plate) in 100 μl culture medium containing 10% or 0.5% v/v FBS. The absorbance at 490 nm was determined with an Elisa plate reader after addition of 20 μl MTS reagent (Promega) and incubation at 37° C. for 2 hours. Growth in normal and limiting serum conditions was not affected by expression of StarD10 (FIG. 6B). Furthermore, no difference in the growth rate was observed when EGF was added to HER14 StarD10 cells grown in 0.5% serum, compared to HER14 Puro control cells. However, StarD10 expression was found to enhance anchorage-independent growth of both NIH3T3 and HER14 cells.

Growth in soft agar was also assessed, as follows: 5×10⁴ for NIH3T3 lines and 2-5×10³ for HER14 lines were plated in duplicate or triplicate in 6 well plates in 2 ml culture medium supplemented with 0.35% Difco agar overlaying a 2 ml 0.7% bottom agar. For stimulation of the EGFR in HER14 lines, 10 ng/ml EGF was added to the top agar. The cells were incubated for four weeks and re-fed on a weekly basis with 300 μl culture medium (with or without EGF for HER14 lines). Cells were stained by addition of 400 μl PBS containing 0.5 mg nitroblue tetrazolium (Sigma) for 24 hours before photographing using a Nikon SMZ-U microscope connected to an Axiocam digital camera (Zeiss). Photographs were analyzed using ImageJ software.

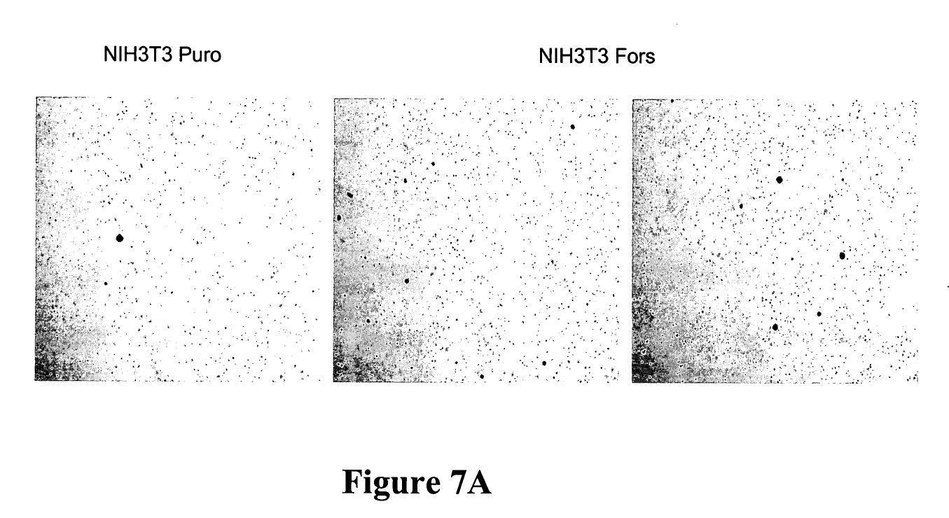

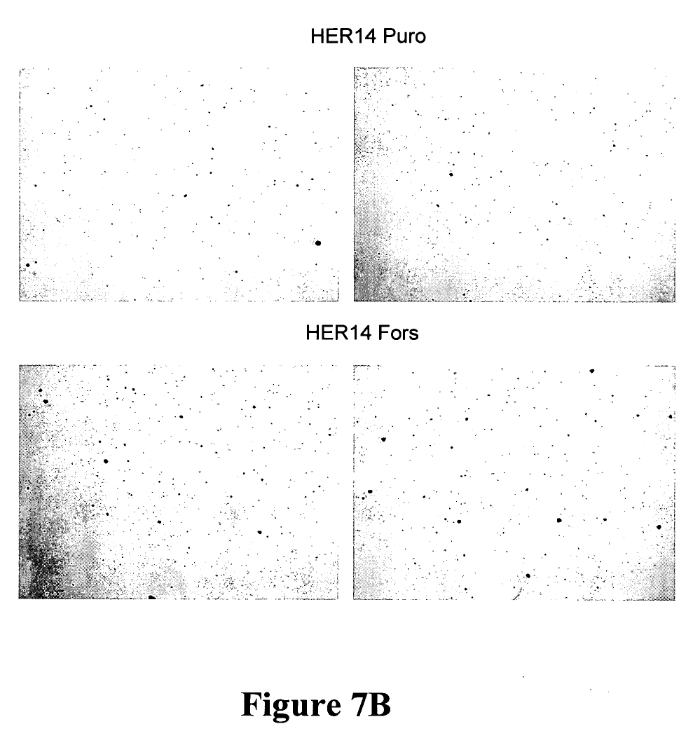

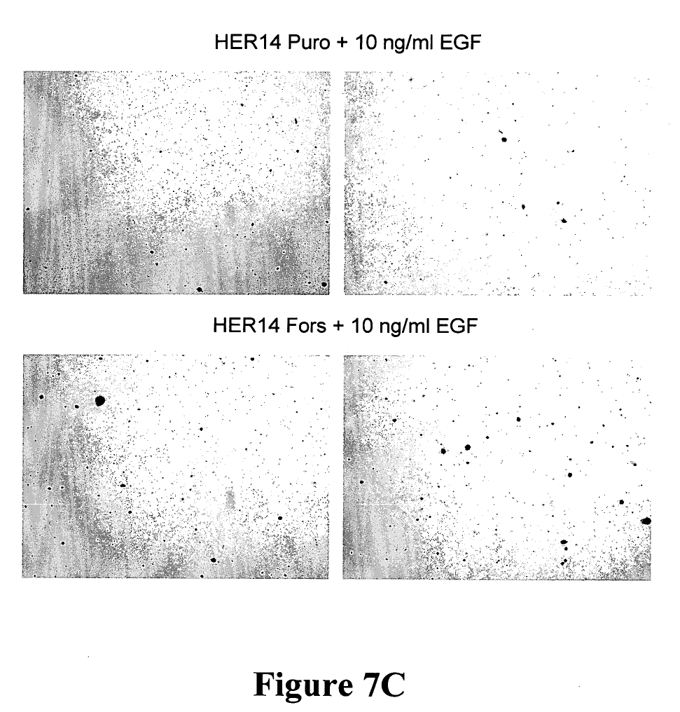

Introduction of StarD10 into NIH3T3 cells allowed outgrowth of few small colonies when 50,000 cells were plated per well (FIG. 7A). HER14 cells are capable of growing in soft agar and colony number and size can be increased by the addition of EGF. Expression of StarD10 in these cells doubled the number of colonies larger than 50 μm in diameter and colonies also reached a larger size (FIGS. 7B and 7C). Taken together, these results suggest that StarD10 specifically affects anchorage independent proliferation, a crucial feature of cellular transformation, and that it functions synergistically with the EGFR.

NIH3T3 cells expressing StarD10 are tested for an ability to form tumors in nude mice. Furthermore, HER14 StarD10 cells are examined to ascertain whether they have a growth advantage in vivo compared HER14 Puro control cells.

### EXAMPLE 12

### StarD10 is Over-Expressed in Primary Human Breast Cancers

A polyclonal antibody, highly specific for StarD10 by Western blotting and immunofluorescence, has been generated. Using this antibody, staining of histological sections of human breast tumors addresses the question of whether StarD10 up-regulation is linked to tumor grade and useful as a prognostic marker. These histological examinations of the StarD10 protein are extended to other tumor types, such as colon carcinoma. Present results indicate that StarD10 up-regulation may be of significance in a subset of human colon carcinoma cell lines. Moreover, screening of a cDNA expression library derived from a human colon tumor patient with autologous serum identified auto-antibodies raised against StarD10 (Scanlan et al., Int. J. Cancer 75: 652-658, 1998), indicating that StarD10 may be a target for antitumoral immune response.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### EXAMPLE 13

### StarD10 is Overexpressed in a Subset of Primary Human Breast Cancers

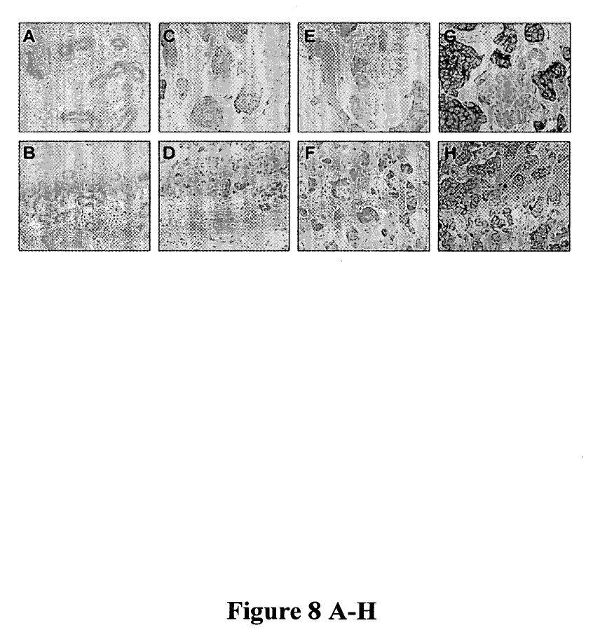

To examine whether StarD10 was upregulated in primary breast cancers, immunohistochemistry was performed on tissue arrays comprising 79 invasive breast cancers. Tumor specimens were scored as either low/negative or moderate/high for expression of StarD10, based on the intensity of staining with affinity-purified StarD10 antibody. A high proportion of tumors (28/79; 35%) yielded moderate to intense staining, and included infiltrating ductal, lobular and mixed carcinomas. Four infiltrating ductal breast carcinomas exhibiting intense staining of the nucleus and cytoplasm are shown in FIG. 8C-F. In contrast, negligible StarD10 expression was observed in normal breast tissue (FIG. 8A and B). Immunostaining with an anti-ErbB2 antibody revealed that of the eight tumors displaying strong membranous staining, four also expressed StarD10 at high levels. Two examples are depicted in FIG. 8 in which the StarD10-expressing tumors E and F correspond to the ErbB2 positive tumors G and H, respectively. While this result indicates that the two genes may be frequently coexpressed at aberrantly high levels, larger numbers of ErbB2 tumors are required to extend this observation.

### EXAMPLE 14

### StartD10 Cooperates with ErbB1 in Agar Assays

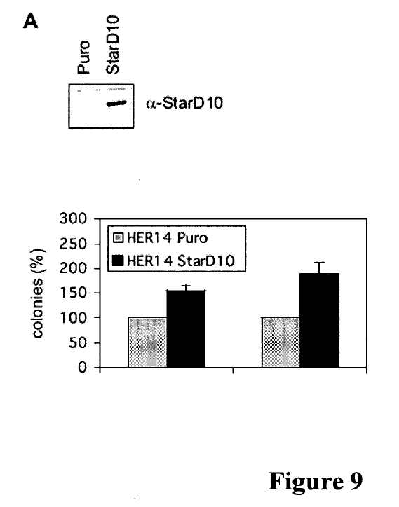

To investigate the effect of overexpression of StarD10 on cellular transformation NIH3T3 and HER14 cells stably expressing StarD10 by retroviral infection were generated. The HER14 line corresponds to NIH3T3 cells that express ErbB1; these cells and parental NIH3T3 cells lack detectable StarD10 by western blotting. Transduction of HER14 cells with StarD10 resulted in expression similar to that seen for StarD10 in breast carcinoma cell lines (FIG. 9A). Overexpression of StarD10 did not alter the norphology of NIH3T3 or HER14 cells, nor was growth altered in either normal and limiting serum conditions. Although StarD10 alone was not sufficient to cause trnasformation of NIH3T3 cells, it was found to enhance anchorage-independent growth of HER14 cells in soft agar. Quantification of digital photographs using image analysis software revealed that StarD10 expression increased both the number and size of colonies. These indicate that StarD10 cooperates with ErbB1 in eliciting anchorage-independent growth.

### EXAMPLE 15

### Downregulation of StarD10 in Breast Carcinoma Cells by RNA Interference Reduces Colony Growth in Soft Agar

To examine the role of StarD10 in breast carcinoma cell lines an siRNA oligo (si240) was used to downregulate expression of StarD10. Transient transfection of MCF7 cells with si240 led to a reduction in StarD10, with levels declining from day 4 until at least day 8 post-transfection. The kinetics of downregulation suggested that StarD10 has a very slow rate of turnover. MCF7 cells with diminished StarD10 levels gave rise to substantially fewer colonies in soft agar. Quantification of colonies greater than 30 μm in size was performed for three independent experiments and showed a 64, 72 and 41% decrease in number. A similar reduction in colony forming ability was observed for SKBR3 cells transfected with si240.

### EXAMPLE 16

### Downregulated StarD10 Expression Impedes Cell Cycle Progression of Breast Carcinoma Cells

The effect of downregulating StarD10 expression in MCF7 cells was examined on their cell cycle profile. Mock- and si240-transfected MCF7 cells were stained with propidium iodide on day 6 post-transfection and analyzed by flow cytometry. Downregulation of StarD10 levels resulted in an increased percentage of cells in G1 phase (72 vs 56%) and a decreased percentage of cells in S phase of the cell cycle (16 vs 31%). Replating ofmock- and si240-transfected MCF7 cells on day 6 post-transfection, allowed us to correlate the proliferative capacity of cells occupied S phase versus only 22% of si240-transfected cells, 24 hrs after replating. A 41% reduction in S phase cells was observed in three independent experiments. This was accompanied by diminished cyclin A levels but no change in that of cyclin D1.

### EXAMPLE 17

### Fluorescent Resonance Energy Transfer (FRET) of Fluorescent Lipid Analogs

Electron spin resonance measurements of spin-labeled lipids in the presence of purified StarD10 protein revealed interaction of StarD10 with phosphatidylcholine (PC) and to a lesser extent with phosphyatidylethanolamine (PE), phosphatidylserine (PS) and sphingomyelin (SM). This interaction appeared to be mediated by the lipid head group since soluble phosphorylcholine competed with the StarD10-PC interaction. The interaction of StarD10 with these phospholipids was further verified using 7-nitro-2,1,3-benzoxadiazol-4-yl (NBD)-labeled fluorescent lipid analogs incorporated into liposomes in the presence of rhodamine-PE. The emission spectrum of NBD overlaps with the excitation of rhodamine, resulting in FRET when lipid analogs are in close proximity. Upon addition of StarD10, an increase in the NBD fluorescence at 535 nm (excitation: 470 nm) was observed for NDB-PC and to a lower degree for PE, PS and SM analogs but not in that of NBD-phosphatidylglycerol. This increase in fluorescence suggests that StarD10 binds these lipids with varying affinity.

Binding of lipids was also shown to lead to conformational changes within the protein as determined by intrinsic fluorescence measurements of StarD10 in the absence and presence of liposomes derived from porcine brain lipids. Tryptophan residues of StarD10 were excited at 280 nm with a maxima fluorescence emission occurring at 335 nm. In the presence of brain lipid liposomes, the intensity of fluorescence was markedly reduced. This indicates a structural change in StarD10 upon interaction with membranes. Moreover, it provides evidence that binding is not restricted to fluorescent lipid analogs but extends to naturally occurring lipids.

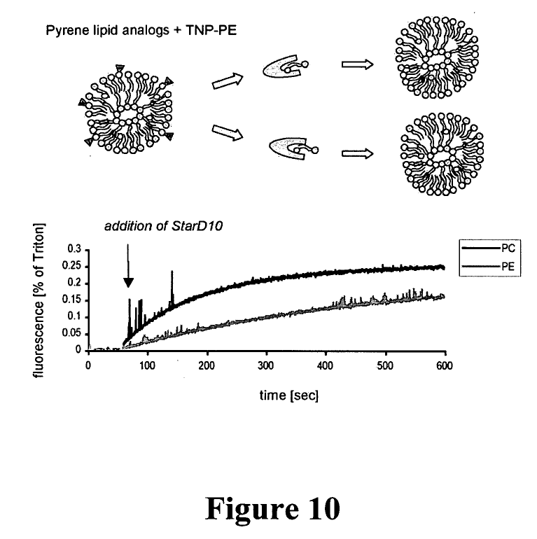

StarD10 not only possessed lipid binding but also lipid transfer activity. In a FRET assay similar to the one described above, pyrene-labeled lipid analogs incorporated into donor liposomes containing quenching amounts of trinitrophenyl-PE (TNP-PE) were transferred to unlabeled acceptor liposomes only upon addition of StarD10 (see FIG. 10). This transfer activity was most pronounced for pyrene-PC but was also observed for PE and SM analogs. Pyrene-labeled glycosylceramide and dehydroergosterol, a cholesterol analog with intrinsic fluorescence, did not serve as substrates, confirming the specificity of the assay. Interestingly, these lipid transfer specificities distinguish StarD10 from the START family member Pctp, which selectively binds and transfers PC suggesting a distinct biological function.

Using a method described by Thiele et al, Nat. Cell. Biol., 2:42-49, 2000, it was demonstrated that StarD10 bond to lipids in vivo. 293T cells were transiently transfected with a StarD10 expression plasmid and labeled with [³H] choline and a photoactivable fatty acid (10-ASA), metabolic precursors of PC. Exposure of cells to UV irradiation then promotes the formation of covalent bonds between labeled PC and any bound proteins. Autoradiography of StarD10 immunoprecipitates separated by SDS-PAGE revealed that StarD10 can indeed engage into a complex with PC in vivo.

Accordingly, StarD10 is a novel PC- and PE-specific lipid transfer protein. Further studies evaluate StarD10 contribution to the activation of cellular phospholipases.

### BIBLIOGRAPHY

Altschul et al., Nucl. Acids Res. 25: 3389-3402. 1997.
Ausubel et al., “Current Protocols in Molecular Biology” John Wiley & Sons Inc, 1994-1998, Chapter 15.
Bandyopadhyay and Temin, Mol. Cell. Biol. 4: 749-754, 1984.
Beaucage and Carruthers, Tetra. Letts. 22. 1859-1862, 1981.
Berglund et al., Biotechnology 11: 916-920, 1993.
Berkner et al., BioTechniques 6; 616-629, 1988.
Berkner, Curr. Top. Microbiol. Immunol. 158: 39-66, 1992.
Bonner and Laskey, Eur. J. Biochem. 46: 83, 1974.
Breakefield and Geller, Mol. Neurobiol. 1: 339-371, 1987.
Buchschacher and Panganiban, J. Virol. 66: 2731-2739, 1982.
Culver, Gene Therapy: A Primer for Physicians, 2^{nd} Ed., Mary Ann Liebert, 1996.
Douillard and Hoffman, Basic Facts about Hybridomas, in Compendium of Immunology Vol. II, ed. by Schwartz, 1981.
Enhancers and Eukaryotic Gene Expression, Cold Spring Harbor Press, Cold Spring Harbour, N.Y., 1983.
Erickson et al., Science 249: 527-533, 1990.
Fiers et al., Nature 273: 113-120, 1978.
Fink et al., Ann. Rev. Neurosci. 19: 265-287, 1996.
Fink et al., Hum. Gene Ther. 3: 11-19, 1992.
Freese et al., Biochem. Pharmacol. 40: 2189-2199, 1990.
Friedman, In: Therapy for Genetic Disease, T. Friedman, Ed., Oxford University Press, pp. 105-121, 1991.
Gorziglia and Kapikian, J. Virol. 66: 4407-4412, 1992.
Guy et al., Proc. Natl. Acad. Sci. USA 89: 10578-10582, 1992.
Helseth et al., J. Virool. 64. 2416-2420, 1990.
Hodgson, Bio/Technology 9: 19-21, 1991.
Hoffmann et al., Proteomics 1: 807-818, 2001.
Jakoby and Pastan (eds.), Cell Culture. Methods in Enzymology, Vol. 58, 1979.
Johnson et al., “Peptide Turn Mimetics” in Biotechnology and Pharmacy, Pezzuto et al., Eds., Chapman and Hall, New York, 1993.
Johnson et al., J. Virol. 66: 2952-2965, 1992.
Kohler and Milstein, European Journal of Immunology 6: 511-519, 1976.
Kohler and Milstein, Nature 256: 495-499, 1975.
Kubo et al., FEBS Lett. 241: 119, 1988.
Kyte and Doolittle, J. Mol. Biol. 157: 105-132, 1982.
Lai et al., Genome Research 10: 703-713, 2000.
Madzak et al., J. Gen. Virol. 73: 1533-1536, 1992.
Mann and Baltimore, J. Virol. 54: 401-407, 1985.
Margolskee, Curr. Top., Microbiol. Immunol. 158: 67-95, 1992.
Marmur and Doty, J. Mol. Biol. 5: 109, 1962.
Matteucci et al., J. Am. Chem. Soc. 103: 3185, 1981.
Miller et al., J. Virol. 62: 4337-4345, 1988.
Miller et al., Mol. Cell. Biol. 5: 431-437, 1985.
Miller, Curr. Top. Microbiol. Immunol. 158: 1-24, 1992.
Moss, Curr. Top. Microbiol. Immunol. 158: 25-38, 1992.
Moss, Proc. Natl. Acad. Sci. USA 93: 11341-11348, 1996.
Munder et al. (Appl. Microbiol. Biotechnol. 52(3): 311-320, 1999.
Muzyczka, Curr. Top. Microbiol. Immunool. 158: 97-129, 1992.
Naldini et al., Science 272: 263-267, 1996.
Ohi et al., Gene 89: 279-282, 1990.
Orita et al., Proc. Nat. Acad. Sci. USA 86: 2776-2770, 1989.
Page et al., J. Virol. 64. 5270-5276, 1990.
Petropoulos et al., J. Viol. 66: 3391-3397, 1992.
Phizicky and Fields, Microbiol. Rev. 59(1): 94-123, 1994
Quantin et al., Proc. Natl. Acad. Sci. USA 89: 2581-2584, 1992.
Remington's Pharmaceutical Sciences, 18^{th} Ed. (Mack Publishing Company, Easton, Pa., U.S.A., 1990.
Rosenfeld et al., Cell 68: 143-155, 1992.
Russell and Hirata, Nature Genetics 18: 323-328, 1998.
Sambrook et al., Molecular Cloning: A Laboratory Manual, 2^{nd} Ed., Cold Spring Harbor Laboratory, Cold Spring Harbor, N.Y., 1989.
Scanlan et al., Int. J. Cancer 75: 652-658, 1998.
Schneider et al., Nature Genetics 18: 180-183, 1998.
Shimada et al., J. Clin. Invest. 88: 1043-1047, 1991
Sorge et al., Mol. Cell. Biol. 4: 1730-1737, 1984.
Stratford-Perricaudet et al., Hum. Gene Ther. 1: 241-256, 1990.
Thiele et al, Nat. Cell. Biol., 2:42-49, 2000.
Visvader, EMBO J. 11: 4557-4564, 1991
Wilkinson et al., Nucleic Acids Res. 20: 2233-2239, 1992.
Young et al., Nat. Biotechnol. 16(10): 946-950, 1998.

1. An isolated nucleic acid molecule comprising a sequence of nucleotides encoding or complementary to a sequence encoding the amino acid sequence set forth in SEQ ID NO:5 or SEQ ID NO:6 or amino acid sequence having at least about 40% similarity to SEQ ID NO:5 or SEQ ID NO:6 after optimal alignment.

2. The isolated nucleic acid molecule of claim 1 wherein the nucleotide sequence encodes the amino acid sequence set forth in SEQ ID NO:5.

3. The isolated nucleic acid molecule of claim 1 wherein the nucleotide sequence encodes the amino acid sequence set forth in SEQ ID NO:6.

4. The isolated nucleic acid molecule of claim 1 comprising a nucleotide sequence set forth in SEQ ID NO:4 or a nucleotide sequence having at least about 40% similarity to SEQ ID NO:4 after optimal alignment or a nucleotide sequence capable of hybridizing to SEQ ID NO:4 or its complementary form under low stringency conditions.

5. The isolated nucleic acid molecule of claim 1 wherein the nucleic acid is selected from the group consisting of: mammalian, insect, reptilian, fish, avian, arachnid, yeast and C. elegans in origin.

6. The isolated nucleic acid molecule of claim 5 wherein the nucleic acid is of mammalian origin.

7. The isolated nucleic acid molecule of claim 5 wherein the nucleic acid is of human origin.

8. The nucleic acid molecule of claim 7 wherein the nucleic acid molecule is from human breast carcinoma cell line SKBR3 as defined in ATCC Catalog No. HTB30.

9. The nucleic acid molecule of claim 1 wherein the nucleic acid is a genomic molecule.

10. The nucleic acid molecule of claim 1 wherein the nucleic acid is a cDNA molecule.

11. An isolated nucleic acid molecule comprising a nucleotide sequence 5′ of the genomic equivalent of SEQ ID NO:4 and comprising a promoter or a functional part thereof.

12. An isolated nucleic acid molecule comprising a heterologous promoter operably linked to SEQ ID NO:4.

13. An isolated vector comprising SEQ ID NO:4 or a homolog thereof.

14. The isolated vector of claim 13 wherein SEQ ID NO:4 or its homolog is operably linked to a promoter.

15. An antisense molecule to SEQ ID NO:4 or its homolog.

16. An expression product of StarD10, said expression product selected from the group consisting of:
(i) an amino acid sequence set forth in SEQ ID NO:5 or an amino acid sequence having at least 40% similarity to SEQ ID NO:5 after optimal alignment;
(ii) an amino acid sequence set forth in SEQ ID NO:5;
(iii) an amino acid sequence set forth in SEQ ID NO:6 or an amino acid sequence having at least 40% similarity to SEQ ID NO:6 after optimal alignment;
(iv) an amino acid sequence set forth in SEQ ID NO:6;
(v) a ribonucleotide sequence corresponding to SEQ ID NO:4 or a nucleotide sequence having at least about 40% similarity to SEQ ID NO:4 after optimal alignment or a nucleotide sequence capable of hybridizing to SEQ ID NO:4 or its complementary form under low stringency conditions; and
(vi) a ribonucleotide sequence corresponding to SEQ ID NO:4.

17. The expression product of claim 16 wherein the product is a protein.

18. The expression product of claim 16 having the amino acid sequence setforth in SEQ ID NO:5 or a homolog, antibody or derivative thereof.

19. The expression product of claim 18 having the amino acid sequence setforth in SEQ ID NO:5.

20. The expression product of claim 16 comprising the amino acid sequence setforth in SEQ ID NO:6 or a homolog, antibody or derivative thereof.

21. The expression product of claim 20 having the amino acid sequence setforth in SEQ ID NO:6.

22. An isolated antibody that specifically binds to a protein of having an amino acid sequence of SEQ ID NO:5 or SEQ ID NO:6.

23. The isolated antibody of claim 22 wherein the antibody is a monoclonal antibody.

24. A method for detecting StarD10 in a biological sample from a subject, said method comprising contacting said biological sample with an antibody specific for StarD10 or its derivatives or homologs for a time and under conditions sufficient for an antibody-StarD10 complex to form, and then detecting said complex.

25. A method for detecting an aberrant cell in a subject or in a biological sample from said subject, said method comprising contacting cells or cell extracts from said subject or said biological sample with an immunointeractive molecule specific for StarD10 or an antigenic portion thereof and screening for the level of immunointeractive molecule-StarD10 complex formations wherein an elevated presence of said complex relative to a normal cell is indicative of an aberrant cell.

26. A method for detecting an aberrant cell in a subject or in a biological sample from said subject, said method comprising screening the level of an expression product of a gene encoding a StarD10, wherein an elevated level of said expression product compared to a normal cell is indicative of an aberrant cell.

27. The method of claim 24 or 25 or 26 wherein the sample is a biopsy comprising biological material selected from the group consisting of: cells, cell extract, tissue, tissue fluid, excretia, circulatory fluid, respiratory fluid, and other material.

28. A method for diagnosing the presence of cancer or cancer-like growth in a subject, said method comprising contacting cells or cell extracts from said subject or a biological sample from said subject with a StarD10-binding effective amount of an antibody having specificity for said StarD10 or an antigenic determinant or epitope thereon and then quantitatively or qualitatively determining the level of a StarD10-antibody complex wherein the presence of elevated levels of said complex compared to a normal cell is indicative of the presence of a cancer.

29. A method for diagnosing the presence of a cancer in a subject, said method comprising obtaining mRNA from cells of said subject or from a biological sample from said subject and optionally generating cDNA and contacting said mRNA or cDNA with a genetic probe capable of hybridizing to and/or amplifying all or part of a StarD10 nucleotide sequence encoding StarD10 or its complementary nucleotide sequence and then detecting the level of said mRNA or cDNA wherein the presence of elevated levels of said mRNA or cDNA compared to normal controls is indicative of the presence of cancer.

30. A method for the treatment of a patient having cancer, said method comprising administering to said human, a cancer cell growth inhibiting-effective amount of an antibody having specificity for human StarD10 protein, wherein said antibody is substantially non-immunogenic and further comprises a cell growth inhibiting or cell killing agent fused, bound or otherwise associated thereto.

31. A method for the treatment of a patient having cancer, said method comprising administering to said patient, a genetic composition comprising a genetic construct which down-regulates expression of a StarD10 gene encoding StarD10.

32. A method for the treatment of a patient having cancer, said method comprising administering to said patient, a genetic composition comprising a genetic construct comprising a nucleotide sequence substantially as set forth in SEQ ID NO:4 or a fragment thereof or a nucleotide sequence having at least about 40% similarity to SEQ ID NO:4 after optimal alignment or a nucleotide sequence capable of hybridizing to SEQ ID NO:4 its complementary form under low stringency conditions.
